(19) United States
(12) Patent Application Publication
(10) Pub. No.: US 2014/0317783 A1
(43) Pub. Date: Oct. 23, 2014

(54) POLYNUCLEOTIDES, POLYPEPTIDES AND METHODS FOR ENHANCING PHOTOSSIMILATION IN PLANTS

(71) Applicant: SYNGENTA PARTICIPATIONS AG, Basel (CH)

(72) Inventors: Michael Nuccio, Research Triangle Park, NC (US); Laura Potter, Research Triangle Park, NC (US); Jonathan Cohn, Research Triangle Park, NC (US)

(73) Assignee: SYNGENTA PARTICIPATIONS AG, Basel (CH)

(21) Appl. No.: 14/355,251

(22) PCT Filed: Nov. 2, 2012
(86) PCT No.: PCT/US2012/063161

§ 371 Date: Apr. 30, 2014

Related U.S. Application Data
(63) Continuation-in-part of application No. PCT/US2011/059123, filed on Nov. 3, 2011.

(51) Int. Cl. C12N 15/82

(52) U.S. Cl. CPC C12N 15/8261
USPC 800/290; 435/320.1; 800/298

(57) ABSTRACT

The present invention relates generally to the field of molecular biology and regards various polynucleotides, polypeptides and methods that may be employed to enhance yield in transgenic plants. Specifically the transgenic plants may exhibit increased yield, increased biomass or increased photoassimilation.

### FIELD OF THE INVENTION

The disclosure relates generally to the field of molecular biology and regards to various polynucleotides, polypeptides and methods of use that may be employed to enhance photoassimilation and yield in transgenic plants. Transgenic plants comprising any one of the polynucleotides or polypeptides described herein may exhibit any one of the traits consisting of increased biomass, increased photoassimilation or increased yield.

### BACKGROUND OF THE INVENTION

The increasing world population and the dwindling supply of arable land available for agriculture fuels the need for research in the area of increasing the efficiency of agriculture. Conventional means for crop and horticultural improvements utilize selective breeding techniques to identify plants having desirable characteristics. However, such selective breeding techniques have several drawbacks, namely that these techniques are often labor intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant's genome. Such technology has the capacity to deliver crops or plants having various improved economic, agronomic or horticultural traits.

### SUMMARY OF THE INVENTION

One embodiment of the invention is an expression cassette comprising at least three polynucleotides selected from the group consisting of a polynucleotide encoding a phosphoenolpyruvate carboxylase, a polynucleotide encoding a fructose-1,6-bisphosphate phosphatase, a polynucleotide encoding a NADP-malate dehydrogenase, a polynucleotide encoding a phosphoribulokinase, and a polynucleotide encoding a pyruvate orthophosphate dikinase. The expression cassette may comprises a polynucleotide encoding a fructose-1,6-bisphosphate phosphatase, a polynucleotide encoding a phosphoribulokinase and a polynucleotide encoding a phosphoenolpyruvate carboxylase or a polynucleotide encoding a fructose-1,6-bisphosphate phosphatase, a polynucleotide encoding a phosphoribulokinase, a polynucleotide encoding a pyruvate orthophosphate dikinase and a polynucleotide encoding a NADP-malate dehydrogenase.

The expression cassette may contain polynucleotides encoding polypeptides having at least 70%, 80%, 90% or 95% identity to SEQ ID NO. 1; SEQ ID NO. 2; SEQ ID NO: 3; SEQ ID NO. 4 or SEQ ID NO: 5. Alternatively, the expression cassette may comprise polynucleotides encoding polypeptides comprising SEQ ID NO. 1, SEQ ID NO. 2, and SEQ ID NO. 3 or SEQ ID NO. 2, SEQ ID NO. 3, SEQ ID NO. 4, and SEQ ID NO. 5. The polynucleotides of the expression cassette may be operably linked to one or more light inducible promoters. The polynucleotides of the expression cassette may also comprise the polynucleotides described in SEQ ID NO. 6; SEQ ID NO. 7 and SEQ ID NO. 8 or SEQ ID NO. 9; SEQ ID NO. 10; SEQ ID NO. 11 and SEQ ID NO. 12.

Additional embodiments include a method for increasing biomass comprising introducing any one of the expression cassette described into a plant cell; growing the plant cell into a plant; and selecting a transgenic plant having increased biomass. The plant may be a C4 plant and could be selected from the group consisting of sugarcane, maize and sorghum. Alternatively, the plant may be maize.

Another embodiment includes a method of making a transgenic plant comprising introducing any of the described expression cassette into a plant; growing the plant cell into a plant; and selecting a plant comprising the expression cassette. The plant may be a C4 plant and could be selected from the group consisting of sugarcane, maize and sorghum. Alternatively, the plant may be maize.

### BRIEF DESCRIPTION OF THE DRAWINGS

FIG. 1 is a plasmid map of 19862 showing SoFBP, SoPRK, and ZmPEPC expression cassettes in a binary vector. “pr-” prefix denotes a promoter; “i-” prefix denotes an intron; “e-” prefix denotes an enhancer; “c-” prefix denotes a coding sequence; “t-” prefix denotes a terminator.

FIG. 2 is a plasmid map of 19863 showing SoFBP, SbPPDK, and SbNADP-MD expression cassettes in a binary vector. “pr-” prefix denotes a promoter; “i-” prefix denotes an intron; “e-” prefix denotes an enhancer; “c-” prefix denotes a coding sequence; “t-” prefix denotes a terminator.

FIG. 3 describes daily photoassimilation and night time respiration in B027A F1 plants. (A) Steady state photoassimilation rate and (B) night time respiration cultivated under closed-chamber conditions. Plants were subject to 16 hour day at 25° C. and 8 hour night at 20° C. Relative humidity was 60%. Atmospheric CO₂ was maintained by metered injection at 400 ppm during the day. Photoassimilation is the daily rate of CO₂ injected to maintain the 400 ppm set point. Night time respiration is the CO₂ released during the night as a function of CO₂ assimilated the previous day. Data are for 40 plants.

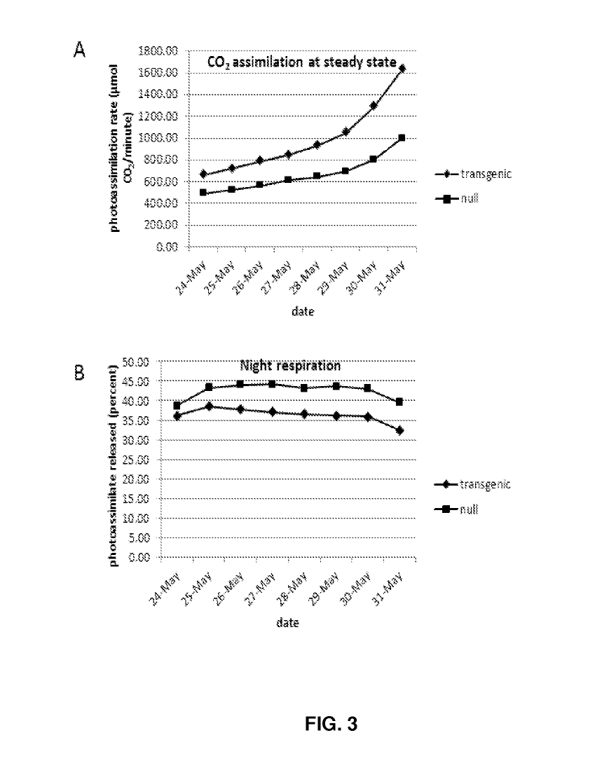

### DETAILED DESCRIPTION OF THE INVENTION

The practice of the present invention will employ, unless otherwise indicated, conventional techniques of botany, microbiology, tissue culture, molecular biology, chemistry, biochemistry, plant quantitative genetics, statistics and recombinant DNA technology, which are within the skill of the art. Such techniques are explained fully in the literature. See, e.g., Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5th ed., Prentice-Hall; Dhringra and Sinclair, (1985) Basic Plant Pathology Methods, CRC Press; Maniatis, et al., (1982) Molecular Cloning: A Laboratory Manual; DNA Cloning, vols. I and II, Glover, ed. (1985); Oligonucleotide Synthesis, Gait, ed. (1984); Nucleic Acid Hybridization, Hames and Higgins, eds. (1984); and the series Methods in Enzymology, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

Unless otherwise defined, all technical and scientific terms used herein have the same meaning as commonly understood by one of ordinary skill in the art to which this invention belongs.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention.

As used herein the singular forms “a”, “and”, and “the” include plural reference unless the context clearly dictates otherwise. Thus, for example, reference to “a vector” is a reference to one or more vectors and includes equivalents thereof known to those skilled in the art.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent.

As used herein, the word “or” means any one member of a particular list and also includes any combination of members on that list.

The terms “comprises”, “comprising”, “includes”, “including”, “having” and their conjugates mean “including but not limited to”. The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

By “microbe” is meant any microorganism (including both eukaryotic and prokaryotic microorganisms), such as fungi, yeast, bacteria, actinomycetes, algae and protozoa, as well as other unicellular structures.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present invention, is implicit in each described polypeptide sequence and incorporated herein by reference.

A “control plant” or “control” as used herein may be a non-transgenic plant of the parental line used to generate a transgenic plant herein. A control plant may in some cases be a transgenic plant line that includes an empty vector or marker gene, but does not contain the recombinant polynucleotide of the present invention that is expressed in the transgenic plant being evaluated. A control plant in other cases is a transgenic plant expressing the gene with a constitutive promoter. In general, a control plant is a plant of the same line or variety as the transgenic plant being tested, lacking the specific trait-conferring, recombinant DNA that characterizes the transgenic plant. Such a progenitor plant that lacks that specific trait-conferring recombinant DNA can be a natural, wild-type plant, an elite, non-transgenic plant, or a transgenic plant without the specific trait-conferring, recombinant DNA that characterizes the transgenic plant. The progenitor plant lacking the specific, trait-conferring recombinant DNA can be a sibling of a transgenic plant having the specific, trait-conferring recombinant DNA. Such a progenitor sibling plant may include other recombinant DNA

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity or ligand/receptor binding is generally at least 30%, 40%, 50%, 60%, 70%, 80% or 90%, preferably 60-90% of the native protein for its native substrate. Conservative substitution tables providing functionally similar amino acids are well known in the art.

The following six groups each contain amino acids that are conservative substitutions for one another:

Alanine (A), Serine (S), Threonine (T);

Aspartic acid (D), Glutamic acid (E);

Asparagine (N), Glutamine (Q);

Arginine (R), Lysine (K);

Isoleucine (I), Leucine (L), Methionine (M), Valine (V) and

Phenylalanine (F), Tyrosine (Y), Tryptophan (W).

See also, Creighton, Proteins, W.H. Freeman and Co. (1984).

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolumn (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the invention, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, by any means, such as, “transfection”, “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, as part of a mini-chromosome or transiently expressed (e.g., transfected mRNA).

As used herein “gene stack” refers to the introduction of two or more genes into the genome of an organism. It may be desirable to stack the genes as described herein with genes conferring insect resistance, disease resistance, increased yield or any other beneficial trait (e.g. increased plant height, etc) known in the art. Alternatively, transgenic plants comprising a gene, polypeptide or polynucleotide as described herein may be stacked with native trait alleles that confer additional traits, such as, improved water use, increased disease resistance and the like. Traits may be stacked by introducing expression cassettes with multiple genes or breeding/crossing plants with one or more traits with other plants containing one or more additional traits.

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “NUE nucleic acid” means a nucleic acid comprising a polynucleotide (“NUE polynucleotide”) encoding a full length or partial length NUE polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise in one case a substantial representation of the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2nd ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement); Sambrook & Russell (2001) Molecular Cloning: A Laboratory Manual., Third Edition, Cold Spring Harbor Laboratory Press, Cold Spring Harbor, N.Y., United States of America. In another instance “nucleic acid library” as defined herein may also be understood to represent libraries comprising a prescribed faction or rather not substantially representing an entire genome of a specified organism. For example, small RNAs, mRNAs and methylated DNA. A nucleic acid library as defined herein might also encompass variants of a particular molecule (e.g. a collection of variants for a particular protein).

As used herein “operably linked” includes reference to a functional linkage between a first sequence, such as a promoter and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

A C4 plant, as defined herein, is one that utilizes the C₄ carbon fixation pathway such that the CO₂ is first bound to a phosphoenopyruvate in a mesophyll cell resulting in the formation of four-carbon compound that is shuttled to the bundle sheath cell where it decarboxylated to liberate the CO₂ to be utilized in the C₃ pathway. Examples of C4 plants include, but are not limited to, members of the Poaceae family (also called Gramineae or true grasses), such as, sugarcane, maize, sorghum, amaranth, millet; members of the sedge family Cyperaceae; and numerous families of Eudicots, including the daisies Asteracae; cabbages Brassicaceae; and spurges Euphorbiaceae.

As used herein, “yield” may include reference to bushels per acre of a grain crop at harvest, as adjusted for grain moisture (15% typically for maize, for example), and the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated. Yield can be affected by many properties including without limitation, plant height, pod number, pod position on the plant, number of internodes, incidence of pod shatter, grain size, efficiency of nodulation and nitrogen fixation, efficiency of nutrient assimilation, carbon assimilation, plant architecture, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill. Yield of a plant of the can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Moreover a bushel of corn is defined by law in the State of Iowa as 56 pounds by weight, a useful conversion factor for corn yield is: 100 bushels per acre is equivalent to 6.272 metric tons per hectare. Other measurements for yield are common practice in the art. In certain embodiments of the invention yield may be increased in stressed and/or non-stressed conditions.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions in most cells.

Any suitable promoter sequence can be used by the nucleic acid construct of the present invention. According to some embodiments of the invention, the promoter is a constitutive promoter, a tissue-specific, or a light inducible promoter.

Suitable constitutive promoters include, for example, CaMV 35S promoter (Odell et al., Nature 313:810-812, 1985); Arabidopsis At6669 promoter (see PCT Publication No. WO04081173A2); maize Ubi 1 (Christensen et al., Plant Mol. Biol. 18:675-689, 1992); rice actin (McElroy et al., Plant Cell 2:163-171, 1990); pEMU (Last et al., Theor. Appl. Genet. 81:581-588, 1991); CaMV 19S (Nilsson et al., Physiol. Plant 100:456-462, 1997); GOS2 (de Pater et al., Plant J November; 2(6):837-44, 1992); ubiquitin (Christensen et al., Plant Mol. Biol. 18: 675-689, 1992); Rice cyclophilin (Bucholz et al., Plant Mol Biol. 25(5):837-43, 1994); Maize H3 histone (Lepetit et al., Mol. Gen. Genet. 231: 276-285, 1992); Actin 2 (An et al., Plant J. 10(1); 107-121, 1996), constitutive root tip CT2 promoter (SEQ ID NO:1535; see also PCT application No. IL/2005/000627) and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable tissue-specific promoters include, but not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed specific genes (Simon, et al., Plant Mol. Biol. 5. 191, 1985; Scofield, et al., J. Biol. Chem. 262: 12202, 1987; Baszczynski, et al., Plant Mol. Biol. 14: 633, 1990), Brazil Nut albumin (Pearson′ et al., Plant Mol. Biol. 18: 235-245, 1992), legumin (Ellis, et al. Plant Mol. Biol. 10: 203-214, 1988), Glutelin (rice) (Takaiwa, et al., Mol. Gen. Genet. 208: 15-22, 1986; Takaiwa, et al., FEBS Letts. 221: 43-47, 1987), Zein (Matzke et al., Plant Mol Biol, 143). 323-32 1990), napA (Stalberg, et al., Planta 199: 515-519, 1996), Wheat SPA (Albani et al, Plant Cell, 9: 171-184, 1997), sunflower oleosin (Cummins, et. al., Plant Mol. Biol. 19: 873-876, 1992)], endosperm specific promoters [e.g., wheat LMW and HMW, glutenin-1 (Mol Gen Genet 216:81-90, 1989; NAR 17:461-2), wheat a, b and g gliadins (EMBO 3:1409-15, 1984), Barley ltrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin Glb-1 (Wu et al., Plant Cell Physiology 39(8) 885-889, 1998), rice alpha-globulin REB/OHP-1 (Nakase et al. Plant Mol. Biol. 33: 513-S22, 1997), rice ADP-glucose PP (Trans Res 6:157-68, 1997), maize ESR gene family (Plant J 12:235-46, 1997), sorgum gamma-kafirin (Plant Mol. Biol 32:1029-35, 1996)], embryo specific promoters [e.g., rice OSH1 (Sato et al., Proc. Nat. Acad. Sci. USA, 93: 8117-8122), KNOX (Postma-Haarsma of al, Plant Mol. Biol. 39:257-71, 1999), rice oleosin (Wu et at, J. Biochem., 123:386, 1998)], and flower-specific promoters [e.g., AtPRP4, chalene synthase (chsA) (Van der Meer, et al., Plant Mol. Biol. 15, 95-109, 1990), LAT52 (Twell et al., Mol. Gen Genet. 217:240-245; 1989), apetala-3; plant reproductive tissues [e.g., OsMADS promoters (U.S. Patent Application 2007/0006344)].

Suitable abiotic stress-inducible promoters include, but not limited to, salt-inducible promoters such as RD29A (Yamaguchi-Shinozalei et al., Mol. Gen. Genet. 236:331-340, 1993); drought-inducible promoters such as maize rab17 gene promoter (Pla et. al., Plant Mol. Biol. 21:259-266, 1993), maize rab28 gene promoter (Busk et. al., Plant J. 11:1285-1295, 1997) and maize Ivr2 gene promoter (Pelleschi et. al., Plant Mol. Biol. 39:373-380, 1999); heat-inducible promoters such as heat tomato hsp80-promoter from tomato (U.S. Pat. No. 5,187,267).

Light inducible promoters have enhanced expression during irradiation with light, while substantially reduced expression or no expression in the absence of light. Examples of light inducible promoter include, but are not limited to, the SSU small subunit gene promoter Berry-Lowe, (1982) J. Mol. Appl. Gen. 1:483-498; pea ribulose-1,5-bisphosphate carboxylase promoter Broglie, R., et al., (1984) Science 224:838-843; Facciotti et al., (1985) “Light-inducible Expression of a Chimeric Gene in Soybean Tissue Transformed with Agrobacterium”, Biotechnology, 3:241-246; Fluhr et al., “Organ-Specific and Light-Induced Expression of Plant Genes”, Science (1986) 232:1106-1112; Lamppa, G., et al. (1985)“Light-regulated and organ-specific expression of a wheat Cab gene in transgenic tobacco”, Nature vol. 316:750-752; Simpson, J., et al., (1985) “Light-inducible and tissue-specific expression of a chimeric gene under control of the 5′-flanking sequence of a pea chlorophyll a/b-binding protein gene”, EMBO Journal vol. 4, No. 11:2723-2729; PSSU gene promoter Herrera-Estrella et al., Nature (1984) 310:115-120; U.S. Pat. No. 5,750,385, and the like.

The term “Enzymatic activity” is meant to include demethylation, hydroxylation, epoxidation, N-oxidation, sulfooxidation, N-, S-, and O-dealkylations, desulfation, deamination, and reduction of azo, nitro, and N-oxide groups. The term “nucleic acid” refers to a deoxyribonucleotide or ribonucleotide polymer in either single- or double-stranded form, or sense or anti-sense, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof.

A “structural gene” is that portion of a gene comprising a DNA segment encoding a protein, polypeptide or a portion thereof, and excluding the 5′ sequence which drives the initiation of transcription. The structural gene may alternatively encode a nontranslatable product. The structural gene may be one which is normally found in the cell or one which is not normally found in the cell or cellular location wherein it is introduced, in which case it is termed a “heterologous gene”. A heterologous gene may be derived in whole or in part from any source known to the art, including a bacterial genome or episome, eukaryotic, nuclear or plasmid DNA, cDNA, viral DNA or chemically synthesized DNA. A structural gene may contain one or more modifications that could affect biological activity or its characteristics, the biological activity or the chemical structure of the expression product, the rate of expression or the manner of expression control. Such modifications include, but are not limited to, mutations, insertions, deletions and substitutions of one or more nucleotides. The structural gene may constitute an uninterrupted coding sequence or it may include one or more introns, bounded by the appropriate splice junctions. The structural gene may be translatable or non-translatable, including in an anti-sense orientation. The structural gene may be a composite of segments derived from a plurality of sources and from a plurality of gene sequences (naturally occurring or synthetic, where synthetic refers to DNA that is chemically synthesized).

“Derived from” is used to mean taken, obtained, received, traced, replicated or descended from a source (chemical and/or biological). A derivative may be produced by chemical or biological manipulation (including, but not limited to, substitution, addition, insertion, deletion, extraction, isolation, mutation and replication) of the original source.

“Chemically synthesized”, as related to a sequence of DNA, means that portions of the component nucleotides were assembled in vitro. Manual chemical synthesis of DNA may be accomplished using well established procedures (Caruthers, Methodology of DNA and RNA Sequencing, (1983), Weissman (ed.), Praeger Publishers, New York, Chapter 1); automated chemical synthesis can be performed using one of a number of commercially available machines.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, an “expression cassette” is a nucleic acid construct, generated recombinantly or synthetically, with a series of specified nucleic acid elements, which permit transcription of a particular nucleic acid in a target cell. The expression cassette can be incorporated into a plasmid, chromosome, mitochondrial DNA, plastid DNA, virus or nucleic acid fragment. Typically, the expression cassette portion of an expression vector includes, among other sequences, a nucleic acid to be transcribed and a promoter.

The terms “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)—500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

“Overexpression” refers to the level of expression in transgenic organisms that exceeds levels of expression in normal or untransformed organisms.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

“Preferred expression”, “Preferential transcription” or “preferred transcription” interchangeably refers to the expression of gene products that are preferably expressed at a higher level in one or a few plant tissues (spatial limitation) and/or to one or a few plant developmental stages (temporal limitation) while in other tissues/developmental stages there is a relatively low level of expression.

The term “transformation” refers to the transfer of a nucleic acid fragment into the genome of a host cell, resulting in genetically stable inheritance. “Transiently transformed” refers to cells in which transgenes and foreign DNA have been introduced (for example, by such methods as Agrobacterium-mediated transformation or biolistic bombardment), but not selected for stable maintenance. “Stably transformed” refers to cells that have been selected and regenerated on a selection media following transformation.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The term “translational enhancer sequence” refers to that DNA sequence portion of a gene between the promoter and coding sequence that is transcribed into RNA and is present in the fully processed mRNA upstream (5′) of the translation start codon. The translational enhancer sequence may affect processing of the primary transcript to mRNA, mRNA stability or translation efficiency. “Visible marker” refers to a gene whose expression does not confer an advantage to a transformed cell but can be made detectable or visible. Examples of visible markers include but are not limited to β-glucuronidase (GUS), luciferase (LUC) and green fluorescent protein (GFP).

“Wild-type” refers to the normal gene, virus, or organism found in nature without any mutation or modification.

As used herein, “plant material,” “plant part” or “plant tissue” means plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, tubers, rhizomes and the like.

As used herein “Protein extract” refers to partial or total protein extracted from a plant part. Plant protein extraction methods are well known in the art.

As used herein “Plant sample” refers to either intact or non-intact (e g milled seed or plant tissue, chopped plant tissue, lyophilized tissue) plant tissue. It may also be an extract comprising intact or non-intact seed or plant tissue.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, and 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40 and 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, such as, at least 50% sequence identity, at least 60% sequence identity, at least 70%, at least 80%, more preferably at least 90% and at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, such as, at least 55%, at least 60%, at least 70%, 80%, 90% and at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

As used herein the phrase “plant biomass” refers to the amount (measured in grams of air-dry or dry tissue) of a tissue produced from the plant in a growing season, which could also determine or affect the plant yield or the yield per growing area.

Increased crop yield is a trait of considerable economic interest throughout the world. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigor may also be important factors in determining yield. In addition it is greatly desirable in agriculture to develop crops that may show increased yield in optimal growth conditions as well as in non-optimal growth conditions (e.g. drought, under abiotic stress conditions). Optimizing the abovementioned factors may therefore contribute to increasing crop yield.

Seed yield is a particularly important trait, since the seeds of many plants are important for human and animal nutrition. Crops such as, corn, rice, wheat, canola and soybean account for over half the total human caloric intake whether through direct consumption of the seeds themselves or through consumption of livestock raised on processed seeds. Plant seeds are also a source of sugars, oils and many kinds of metabolites used in various industrial processes. Seeds consist of an embryo (the source of new shoots and roots) and an endosperm (the source of nutrients for embryo growth during germination and during early growth of seedlings). The development of a seed involves many genes, and requires the transfer of metabolites from the roots, leaves and stems into the developing seed. The endosperm assimilates the metabolic precursors of carbohydrates, oils and proteins and synthesizes them into storage macromolecules to fill out the grain.

In some instances plant yield is relative to the amount of plant biomass a particular plant may produce. A larger plant with a greater leaf area can typically absorb more light, nutrients and carbon dioxide than a smaller plant and therefore will likely gain a greater weight during the same period (Fasoula & Tollenaar 2005 Maydica 50:39). Increased plant biomass may also be highly desirable in processes such as the conversion of biomass (e.g. corn, grasses, sorghum, cane) to fuels such as for example ethanol or butanol.

The ability to increase plant yield would have many applications in areas such as agriculture, the production of ornamental plants, arboriculture, horticulture, biofuel production, pharmaceuticals, enzyme industries which use plants as factories for these molecules and forestry. Increasing yield may also find use in the production of microbes or algae for use in bioreactors (for the biotechnological production of substances such as pharmaceuticals, antibodies, vaccines, and fuel or for the bioconversion of organic waste) and other such areas.

Plant breeders are often interested in improving specific aspects of yield depending on the crop or plant in question, and the part of that plant or crop which is of relative economic value. For example, a plant breeder may look specifically for improvements in plant biomass (weight) of one or more parts of a plant, which may include aboveground (harvestable) parts and/or harvestable parts below ground. This is particularly relevant where the aboveground parts or below ground parts of a plant are for consumption. For many crops, particularly cereals, an improvement in seed yield is highly desirable. Increased seed yield may manifest itself in many ways with each individual aspect of seed yield being of varying importance to a plant breeder depending on the crop or plant in question and its end use.

It would be of great advantage to a plant breeder to be able to pick and choose the aspects of yield to be altered. It may also be highly desirable to be able to pick a gene suitable for altering a particular aspect of yield (e.g. seed yield, biomass weight, water use efficiency, and yield under stress conditions). For example an increase in the fill rate, combined with increased thousand kernel weight would be highly desirable for a crop such as corn. For rice and wheat a combination of increased fill rate, harvest index and increased thousand kernel weight would be highly desirable.

Various systems, computer program products and methods for using a model of biological process can predict candidate components such as genes and/or combinations of genes that enhance the biological process. For example, please see the methods as disclosed in WO2012/061585, published on 10 May 2012 and hereby incorporated by reference. One may select a candidate component based on the phenotypic outcome and the determined sensitivity for the purpose of producing a biological product that exhibits or will exhibit the phenotypic outcome. For example, a candidate gene may be selected based on a phenotypic outcome in which the gene is predicted to cause and based on the determined sensitivity. In this manner, a single candidate gene that is relatively insensitive to variations to the optimal expression level may cause the predicted phenotypic outcome or a phenotypic outcome that is acceptably close (based on a predefined difference) to the predicted phenotypic outcome even when the optimal expression levels are not achieved in the biological product during, for example, laboratory experimentation and/or manufacturing.

In one embodiment, the polynucleotide sequence of the selected candidate gene(s) identified by the invention can be synthesized or isolated and introduced into expression cassettes, which contain genetic regulatory elements to target the expression level and cell type(s). In one embodiment, at least one expression cassette may be introduced into a binary vector and transformed into plants. The sensitivity and actual phenotypic outcome can then be determined. As described in the examples below, one embodiment uses the invention to identify three or four candidate genes which are introduced into expression cassettes and transformed into plants using methods known to one skilled in the art. The examples also describe known methods for measuring the phenotypic outcome of the transgenic plants.

One embodiment of the invention includes an expression cassette, cell, or plant comprising alone or in any combination a phosphoenolpyruvate carboxylase (PEPC, EC 4.1.1.31), a fructose-1,6-bisphosphate phosphatase (FBP, EC 3.1.3.11), a NADP-malate dehydrogenase (NADPMD, EC 1.1.1.82), a phosphoribulokinase (PRK, EC 2.7.1.19), and a pyruvate, orthophosphate dikinase (PPDK, EC 2.7.9.1). Sequence information on numerous PEPC, FBP, NADPMD, PRK or PPDK genes can be found in the literature or by querying various databases available, such as, The BRENDA database (brenda.enzymes.org).

Another embodiment of the invention includes an expression cassette, cell or plant comprising any two genes in combination comprising a phosphoenolpyruvate carboxylase (PEPC), a fructose-1,6-bisphosphate phosphatase (FBP), a NADP-malate dehydrogenase (NADPMD), a phosphoribulokinase (PRK), and a pyruvate, orthophosphate dikinase (PPDK).

Yet another embodiment of the invention includes an expression cassette, cell or plant comprising any three genes in combination comprising a phosphoenolpyruvate carboxylase (PEPC), a fructose-1,6-bisphosphate phosphatase (FBP), a NADP-malate dehydrogenase (NADPMD), a phosphoribulokinase (PRK), and a pyruvate, orthophosphate dikinase (PPDK). In a particular embodiment, expression cassettes, cells or plant comprising a fructose-1,6-bisphosphate phosphatase (FBP), a phosphoribulokinase (PRK) and a phosphoenolpyruvate carboxylase (PEPC).

Yet another embodiment of the invention includes an expression cassette, cell or plant comprising any four genes in combination comprising a phosphoenolpyruvate carboxylase (PEPC), a fructose-1,6-bisphosphate phosphatase (FBP), a NADP-malate dehydrogenase (NADP-MD), phosphoribulokinase (PRK), and a pyruvate, orthophosphate dikinase (PPDK). In a particular embodiment, expression cassettes, cells or plant comprising a fructose-1,6-bisphosphate phosphatase (FBP), a phosphoribulokinase (PRK), a NADP-malate dehydrogenase (NADP-MD) and a phosphoenolpyruvate carboxylase (PEPC).

Yet another embodiment of the invention includes an expression cassette, cell or plant comprising a phosphoenolpyruvate carboxylase (PEPC), a fructose-1,6-bisphosphate phosphatase (FBP), a NADP-malate dehydrogenase (NADP-MD), phosphoribulokinase (PRK), and a pyruvate, orthophosphate dikinase (PPDK).

One embodiment of the invention can also include an expression cassette, cell or plant comprising SEQ ID NO. 6, SEQ ID NO. 7, and SEQ ID NO. 8 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 6, SEQ ID NO. 7, and SEQ ID NO. 8.

Another embodiment of the invention includes an expression cassette, cell or plant comprising any two of the sequences SEQ ID NO. 6, SEQ ID NO. 7, and SEQ ID NO. 8 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 6, SEQ ID NO. 7, and SEQ ID NO. 8.

Yet another embodiment of the invention includes an expression cassette, cell or plant comprising one of the sequences SEQ ID NO. 6, SEQ ID NO. 7, and SEQ ID NO. 8 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 6, SEQ ID NO. 7, and SEQ ID NO. 8.

The present invention includes an expression cassette, cell or plant comprising at least one of the sequences SEQ ID NO. 6, SEQ ID NO. 7, or SEQ ID NO. 8 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 6, SEQ ID NO. 7, and SEQ ID NO. 8.

Yet another embodiment of the invention includes an expression cassette, cell or plant comprising the sequences SEQ ID NO. 9, SEQ ID NO. 10, and SEQ ID NO. 11, and SEQ ID NO. 12 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 9, SEQ ID NO. 10, SEQ ID NO. 11 and SEQ ID NO. 12.

Another embodiment of the invention includes an expression cassette, cell, plant, or mammal comprising two of the sequences SEQ ID NO. 9, SEQ ID NO. 10, and SEQ ID NO. 11, and SEQ ID NO. 12 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 9, SEQ ID NO. 10, SEQ ID NO. 11 and SEQ ID NO. 12.

One embodiment of the invention also includes an expression cassette, cell, plant, or mammal comprising one of the sequences SEQ ID NO. 9, SEQ ID NO. 10, and SEQ ID NO. 11, and SEQ ID NO. 12 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 9, SEQ ID NO. 10, SEQ ID NO. 11 and SEQ ID NO. 12.

An embodiment of the invention includes an expression cassette, cell, plant or mammal plant comprising at least one of the sequences SEQ ID NO. 9, SEQ ID NO. 10, and SEQ ID NO. 11, and SEQ ID NO. 12 or polynucleotides have 50, 60, 70, 75, 80, 85, 90, 91, 92, 93, 94, 95, 96, 97, 98, 99, or 100% identity or polynucleotides having capable of hybridizing under low, medium or high stringent hybridization conditions to SEQ ID NO. 9, SEQ ID NO. 10, SEQ ID NO. 1 land SEQ ID NO. 12.

The foregoing examples described herein are for illustrative purposes only and are not intended to be limiting. Implementations of the invention may be made in hardware, firmware, software, or any suitable combination thereof. Implementations of the invention may also be implemented as instructions stored on a machine readable medium, which may be read and executed by one or more processors. A tangible machine-readable medium may include any tangible, non-transitory, mechanism for storing or transmitting information in a form readable by a machine (e.g., a computing device). For example, a tangible machine-readable storage medium may include read only memory, random access memory, magnetic disk storage media, optical storage media, flash memory devices, and other tangible storage media. Intangible machine-readable transmission media may include intangible forms of propagated signals, such as carrier waves, infrared signals, digital signals, and other intangible transmission media. Further, firmware, software, routines, or instructions may be described in the above disclosure in terms of specific exemplary implementations of the invention, and performing certain actions. However, it will be apparent that such descriptions are merely for convenience and that such actions in fact result from computing devices, processors, controllers, or other devices executing the firmware, software, routines, or instructions.

Implementations of the invention may be described as including a particular feature, structure, or characteristic, but every aspect or implementation may not necessarily include the particular feature, structure, or characteristic. Further, when a particular feature, structure, or characteristic is described in connection with an aspect or implementation, it will be understood that such feature, structure, or characteristic may be included in connection with other implementations, whether or not explicitly described. Thus, various changes and modifications may be made to the provided description without departing from the scope or spirit of the invention. As such, the specification and drawings should be regarded as exemplary only, and the scope of the invention to be determined solely by the appended claims.

The following Examples provide illustrative embodiments. In light of the invention and the general level of skill in the art, those of skill will appreciate that the following Examples are intended to be exemplary only and that numerous changes, modifications, and alterations can be employed without departing from the scope of the presently claimed subject matter.

Unless indicated otherwise, The cloning steps carried out for the purposes of the present invention, such as, for example, restriction cleavages, agarose gel electrophoresis, purification of DNA fragments, linking DNA fragments, transformation of E. coli cells, growing bacteria, and sequence analysis of recombinant DNA, are carried out as described by Sambrook, et. al., supra.

### Summary of the Sequence Listing

SEQ ID NO: 1 depicts a polypeptide sequence, Zea mays phosphoenolpyruvate carboxylase

SEQ ID NO: 2 depicts a polypeptide sequence, Spinacia oleracea fructose-1,6-bisphosphate phosphatase
SEQ ID NO: 3 depicts a polypeptide sequence, Spinacia oleracea phosphoribulokinase
SEQ ID NO: 4 depicts a polypeptide sequence, Spinacia oleracea NADP-malate dehydrogenase
SEQ ID NO: 5 depicts a polypeptide sequence, Sorghum bicolor engineered pyruvate, orthophosphate dikinase
SEQ ID NO: 6 depicts a polynucleotide sequence, SoFBP in expression cassette ZmPRK-1
SEQ ID NO: 7 depicts a polynucleotide sequence, SoPRK in expression cassette ZmSBP
SEQ ID NO: 8 depicts a polynucleotide sequence, ZmPEPC in expression cassette ZmPGK
SEQ ID NO: 9 depicts a polynucleotide sequence, SoFBP in expression cassette ZmPRK-2
SEQ ID NO: 10 depicts a polynucleotide sequence, SoPRK in expression cassette ZmNADPME
SEQ ID NO: 11 depicts a polynucleotide sequence, SbPPDK in expression cassette ZmPEPC
SEQ ID NO: 12 depicts a polynucleotide sequence, SbNADP-MD in expression cassette ZmPGK

### Example 1

### Identify Candidates

This example describes a genetic engineering strategy to enhance photoassimilation in maize and other NADP malic-type C4 species. A computer model output was organized into 3 and 4 gene combination solutions. A 3-gene and a 4-gene combination were each selected for trait development. To implement this trait, The BRENDA database (brenda.enzymes.org) was queried for sequence information on phosphoenolpyruvate carboxylase (PEPC, EC 4.1.1.31), fructose-1,6-bisphosphate phosphatase (FBPase, EC 3.1.3.11), phosphoribulokinase (PRK, EC 2.7.1.19), NADP-malate dehydrogenase (NADPME, EC 1.1.1.82) and pyruvate, orthophosphate dikinase (PPDK, EC 2.7.9.1). This analysis provided protein sequence for enzymes that have been functionally characterized. Information from the database was used to obtain the protein sequence for PEPC from Zea mays, FBPase from Spinacia oleracea, phosphoribulokinase from Spinacia oleracea, and NADP-malate dehydrogenase from Sorghum bicolor. Briefly, reference information was used to identify candidates supported by functional characterization data. Each sequence had to be supported by enzyme activity evidence. The protein sequence data are provided (SEQ ID NO 1-4). Despite the available information and number of publications, the public sequence data for maize PPDK was found to be incomplete. Therefore, the Sorghum bicolor PPDK gDNA sequence was defined using public data. The sorghum gDNA and cDNA sequence were pulled from the sorghum genome database using the maize PPDK cDNA and protein sequence as the queries. The sorghum cDNA was expanded through alignment with corresponding ESTs. The sequences were compiled into a contig that was broken into exons and aligned with the gDNA. There are 19 exons, and all but one defines introns bordered by GT . . . AG sequence. There were several places where sorghum PPDK gDNA and cDNA sequence diverged; in most instances the cDNA sequence was substituted for the gDNA sequence. The maize and sorghum protein sequences were also aligned and used to further refine the gDNA sequence. Finally, the Flaveria brownie PPDK residue substitutions were introduced. The result is the SbPPDK-engineered sequence, SEQ ID NO 5. The gDNA sequence was also modified to silence XhoI, SanDI, NcoI, SacI, RsrII, and XmaI restriction endonuclease sites by base substitution. An NcoI site was added at the translation start codon and a SacI site was added after the translation stop codon.

### Example 2

### Regulatory Sequences to Target Candidate Gene Expression

Once candidate genes were identified, regulatory sequences were selected to target expression of the candidate genes to the appropriate cell type. A series of plant expression cassettes were designed to deliver robust trait gene expression in either mesophyll or bundle sheath cells. A combination of proteomic data (Majeran, W., et. al. (2005) Plant Cell 17: 3111-3140) and expression profiling data was used to identify candidate regulatory sequences based on the expression patterns of genes of interest, and six novel expression cassettes were identified (Coneva V, et. al. (2007) J of Exp Botany 58:3679-3693). Each cassette is composed of promoter and terminator sequences. The promoter consists of 5′-non-transcribed sequence, the first intron, and a 5′-untranslated sequence that is made up of the first and part of the second exon. In addition the promoter terminates with a translational enhancer derived from the tobacco mosaic virus omega sequence (Gallie, D. R., Walbot, V. (1992) Nucleic Acids Res 20(17): 4631-4638) and a maize-optimized sequence (Kozak, M. (2002) Gene 299: 1-34). The terminator consists of 3′-untranslated sequence starting just after the translation stop codon and 3′-non-transcribed sequence.

Specific base substitutions were made to eliminate internal XhoI, SanDI, NcoI, SacI, RsrII and XmaI restriction endonuclease sites. In addition base substitutions were used to eliminate ATGs and insert stop codons in the 5′-untranslated sequence. The promoters were flanked with XhoI/SanDI at the 5′-end and NcoI on the 3′-end. The terminators were flanked with SacI at the 5′-end and RsrII/XmaI on the 3′-end. Cassettes were cloned sequentially as RsrII/SanDI fragments into binary vector cut with RsrII. Cassettes are summarized in the Table below, which includes a reference to the relevant SEQ ID NO.

TABLE 1

| | | | Expression |
|---|---|---|---|
| | Gene | Maize Gene | in |
| Candidate | Name | Chip probe | Cell Type |
| phosphribulokinase- | ZmPRK-2 | Zm000129_at | Bundle sheath |
| 2 | | | |
| phosphribulokinase | ZmPRK-1 | Zm003395_at | Bundle sheath |
| sedoheptulose-1,7- | ZmSBP | Zm009018_at | Bundle sheath |
| bisphosphatase | | | |
| phosphoglycerate | ZmPGK | Zm008627_at | Mesophyll |
| kinase | | | |
| NADP-dependent | ZmNADPME | MZENDMEX_at | Mesophyll |
| malic enzyme | | | |

### Example 3

### Expression Cassettes and Combinations

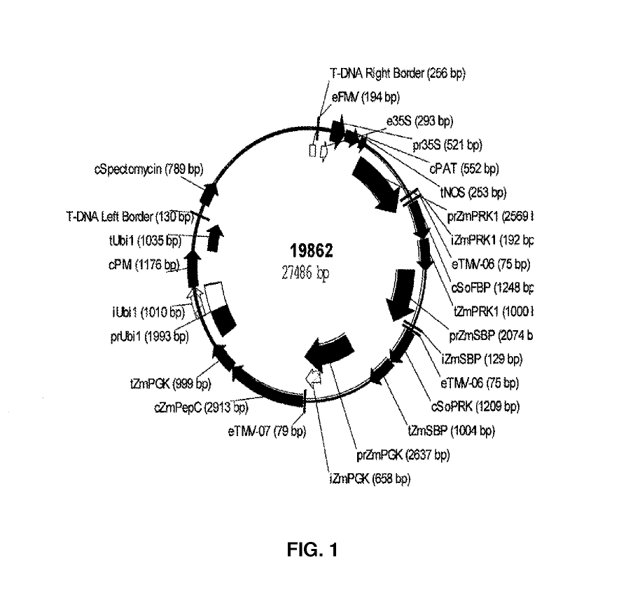

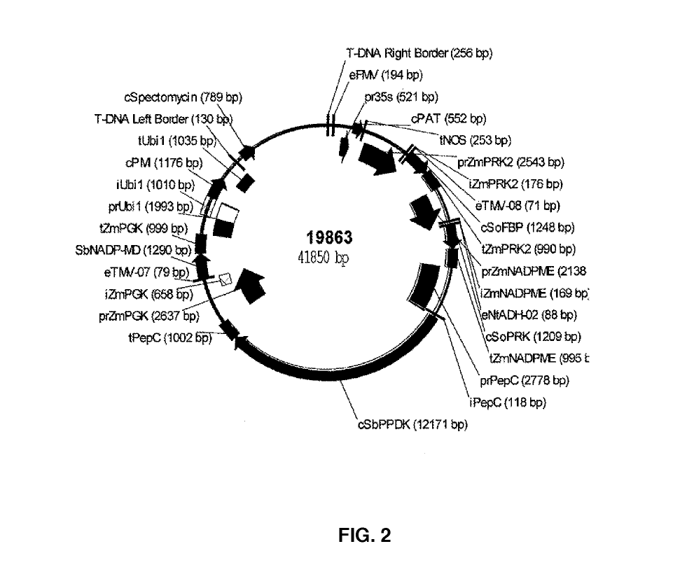

A three-gene and a four-gene expression cassette binary vector containing the candidate genes selected by the method of the present invention will each be used to reduce the C4 photosynthesis model output to practice. The three gene C4 photosynthesis enhancement construct is shown in Table 2; the four gene C4 photosynthesis enhancement construct is shown in Table 3. The gene number indicates order, starting at the right border of the T-DNA and extending to the left border. The three gene binary vector is 19862 and is shown in FIG. 1. The four gene binary vector is 19863 and is shown in FIG. 2.

TABLE 2

| | | Ex- | Transla- | SEQ |
|---|---|---|---|---|
| Num- | Trait | pression | tional | ID |
| ber | Gene | Cassette | enhancer | NO |
| 1 | Fructose-1,6-bisphosphatase | ZmPRK-1 | eTMV-06 | 6 |
| | (SoFBP) | | | |
| 2 | phosphoribulokinase (SoPRK) | ZmSBP | eTMV-06 | 7 |
| 3 | phosphoenolpyruvate | ZmPGK | eTMV-07 | 8 |
| | carboxylase (ZmPEPC) | | | |

TABLE 3

| | | Ex- | Transla- | SEQ |
|---|---|---|---|---|
| Num- | Trait | pression | tional | ID |
| ber | Gene | Cassette | enhancer | NO |
| 1 | Fructose-1,6- | ZmPRK-2 | eTMV-08 | 9 |
| | bisphosphatase (SoFBP) | | | |
| 2 | phosphoribulokinase | ZmNADPME | eNtADH-02 | 10 |
| | (SoPRK) | | | |
| 3 | pyruvate, orthophosphate | ZmPEPC | | 11 |
| | dikinase (SbPPDK) | | | |
| 4 | NADP-malate | ZmPGK | eTMV-07 | 12 |
| | dehydrogenase (SbNADP- | | | |
| | MD) | | | |

### Example 4

### Plant Transformation

Constructs 19862 and 19863 were used for Agrobacterium-mediated maize transformation. Transformation of immature maize embryos was performed essentially as described in Negrotto et al., 2000, Plant Cell Reports 19: 798-803. For this example, all media constituents were essentially as described in Negrotto et al., supra. However, various media constituents known in the art may be substituted.

The genes used for transformation were cloned into a vector suitable for maize transformation. Vectors used in this example contain the phosphomannose isomerase (PMI) gene for selection of transgenic lines (Negrotto et al., supra), as well as the selectable marker phosphinothricin acetyl transferase (PAT) (U.S. Pat. No. 5,637,489). Briefly, Agrobacterium strain LBA4404 (pSB1) containing a plant transformation plasmid was grown on YEP (yeast extract (5 g/L), peptone (10 g/L), NaCl (5 g/L), 15 g/1 agar, pH 6.8) solid medium for 2-4 days at 28° C. Approximately 0.8×10⁹ Agrobacterium were suspended in LS-inf media supplemented with 100 M As (Negrotto et al., supra). Bacteria were pre-induced in this medium for 30-60 minutes.

Immature embryos from A188 or other suitable genotype were excised from 8-12 day old ears into liquid LS-inf+100 M As. Embryos were rinsed once with fresh infection medium. Agrobacterium solution is then added and embryos were vortexed for 30 seconds and allowed to settle with the bacteria for 5 minutes. The embryos were then transferred scutellum side up to LSAs medium and cultured in the dark for two to three days. Subsequently, between 20 and 25 embryos per petri plate were transferred to LSDc medium supplemented with cefotaxime (250 mg/1) and silver nitrate (1.6 mg/1) and cultured in the dark for 28° C. for 10 days.

Immature embryos, producing embryogenic callus were transferred to LSD1M0.5S medium. The cultures were selected on this medium for about 6 weeks with a subculture step at about 3 weeks. Surviving calli were transferred to Reg1 medium supplemented with mannose. Following culturing in the light (16 hour light/8 hour dark regiment), green tissues were then transferred to Reg2 medium without growth regulators and incubated for about 1-2 weeks. Plantlets were transferred to Magenta GA-7 boxes (Magenta Corp, Chicago Ill.) containing Reg3 medium and grown in the light.

Plants were assayed for PMI, PAT, one candidate gene coding sequence and vector backbone by TaqMan. Plants that were positive for PMI, PAT and the candidate gene coding sequence and negative for vector backbone were transferred to the greenhouse. Expression for all trait expression cassettes was assayed by qRT-PCR. Fertile, single copy events were identified and transferred to the greenhouse.

### Example 5

### Evaluation of Transgenic Plants Expressing Candidate Genes

Plant photoassimilation can be assessed in several ways. The following prophetic example described how the transgenic plants described above will be measured for changes in plant photoassimilation. First plant growth between hemizygous trait positive and null seedlings can be compared in V3 seedlings. In this assay, approximately 60 B1 plants are germinated in 4.5 inch pots and genotyped. About 17 days after germination the pot soil is saturated with water and the soil surface is sealed to prevent evaporation. Some seedlings are sacrificed to determine shoot mass (in both fresh and dry weight) at time zero. Pot mass is recorded daily to assess plant water demand. After 7 days shoots are harvested and weighed (both fresh and dry weight). Plant water utilization is corrected using a pot with no plant to report natural water loss. This protocol enables plant growth and water utilization to be compared between trait positive and null groups. Improved photoassimilation may enable the trait positive plants to accumulate more aerial biomass relative to null plants.

A second method is to measure photoassimilation using an infrared gas analysis (IRGA) instrument. For example a CIRAS-2 IRGA device can be fixed to a tripod to gently clamp the gas exchange cuvette to leaves and minimize data noise generated by plant handling. Stomatal aperture is very sensitive to touch and plant movement. The environment applied to the leaf patch can be programmed to mimic a growth chamber environment (400 μmol CO₂; 26° C.; ambient humidity) to assess steady-state photosynthesis under standard growth conditions. In this way photoassimilation between trait positive and null plants can be directly compared.

Although IRGA is a powerful and common tool to assess photosynthetic activity (e.g. A/Ci curves), it has some caveats. First, it only assays a small leaf patch and does not provide information on whole-plant and canopy-level photosynthesis, which are ultimately required to determine trait function in an agronomic context. Second, many measurements are needed to determine A throughout plant development. Third, the general state of the photosynthetic apparatus depends on which leaf is assayed and when it is assayed; there is variability throughout the plant. Finally, it is an invasive technique requiring direct contact with the leaf. A component of the data generated is leaf response to the instrument. Taken together this creates high (10-15%) coefficients of variation. Hence, it may not be possible to detect small, but significant changes in photoassimilation using this device.

To bypass these limitations, large hypobaric chambers such as the chambers at the Controlled Environment Systems Research Facility at the University of Guelph, Ontario (Wheeler, R. M., et. al. (2011) Adv Space Res 47:1600-1607) can be used to monitor with high precision plant CO₂ demand, night time respiration and transpiration of a 30-40 plant population for periods lasting up to several weeks.

### Example 6

### Production of Transgenic Maize with Constructs 19862 and 19863

Transgenic maize events were produced according to Example 4, using binary vectors 19862 and 19863. A total of 32 single-copy, backbone free 19862 events were identified. A total of 22 single-copy, backbone free 19863 events were identified. Messenger RNA produced from each transgene was measured in seedling leaf tissue by qRT-PCR. The qRT-PCR data are reported as the ratio of the gene-specific (coding sequence) signal to that of the endogenous control signal times 1000. Data in the Table below show that all the trait expression cassettes function to produce trait transcript in leaf as expected. Data for the constitutive expression cassettes are included as a benchmark for signal strength. It should be noted that the constitutive cassettes are active in far more leaf cells than the trait cassettes which are restricted to either mesophyll or bundle sheath cells.

TABLE 4

| | Event | Regulatory | Coding | | Relative expression | |
|---|---|---|---|---|---|---|
| Vector | number | sequence | sequence | Target cell | mean | stdev |
| 19862 | 32 | 35S/NOS | PAT | All | 12200 | 9880 |
| | | ZMPRK1 | SoFBP | bundle sheath | 188 | 241 |
| | | ZmSBP | SoPRK | bundle sheath | 214 | 149 |
| | | ZmPGK | ZmPEPC | mesophyll | 1240 | 720 |
| | | ZmUbi1 | PMI | All | 6990 | 6120 |
| 19863 | 22 | 35S/NOS | PAT | All | 13100 | 12900 |
| | | ZMPRK2 | SoFBP | bundle sheath | 484 | 276 |
| | | ZmNADPME | SoPRK | bundle sheath | 10200 | 5980 |
| | | ZmPEPC | SbPPDK | mesophyll | 3860 | 2820 |
| | | ZmPGK | SbNADP-MD | mesophyll | 2270 | 1920 |
| | | ZmUbi1 | PMI | All | 4850 | 3200 |

T0 seedling leaf tissue was sampled for qRT-PCR analysis roughly two weeks after transfer to soil (V3). Gene-specific TaqMan probes were used to determine transcript abundance. Data are reported relative to EF1A transcript, the internal control. Each event was assayed in quadruplicate. Data are the mean±standard deviation for each construct.

### Example 7

### Seedling Biomass Accumulation in a Growth Chamber

Seedling growth can be used to determine if a trait has the potential to cause yield drag. We used this assay to determine if either the 19862 or 19863 traits reduced plant growth. Back-crossed seed were germinated and seedlings were evaluated in a growth chamber according to Example 5. Seedlings for each event were genotyped to establish trait segregation and organize transgenic and null groups. Trait segregation was confirmed as 1 null: 1 hemizygote, as expected, for each event. Data in the Table below summarize the results of several assays. For each event, growth of the transgenic seedlings could not be distinguished from the null seedlings. This indicates the trait is not impeding growth. The wild type plants are included as a benchmark. It should be noted that plants one generation removed from a parent regenerated through tissue culture tend to grow slower than non-transformed or wild type plants. The mean data suggest that the 19862 plants may be growing slower than the wild type plants but the difference is not statistically significant.

TABLE 5

Shoot final dry
weight (grams)

| Vector | Events | Genotype | Ave | StDev |
|---|---|---|---|---|
| 19862 | 6 | null | 2.99 | 0.65 |
| | | transgenic | 2.80 | 0.57 |
| 19863 | 1 | null | 3.70 | 1.28 |
| | | transgenic | 3.28 | 1.14 |
| AX5707 | 1 | wild type | 3.45 | 0.78 |

Transgenic B1 seed were germinated in 4.5 inch pots and genotyped. Plants for each event were organized into transgenic and null groups which were grown in a growth chamber. Shoots were harvested 24 days after planting. Shoots were dried in an oven at 89° C. for 5 days then weighed. Data report the mean±standard deviation for each construct.

### Example 8

### Evaluation of 19862 Events in Closed Chambers

Closed growth chambers can be used to accurately assess whole plant photoassimilation and respiration. Hybrid seed that segregate for the 19862 trait were made for two events, and evaluated in large hypobaric chambers at the Controlled Environment Systems Research Facility at the University of Guelph as described in Example 5. Seed were germinated, genotyped and organized into trait positive and trait negative groups of 40 plants. Ten seedlings per group were weighed at the beginning of the experiment. Each group was placed in a hypobaric chamber and grown for 4 weeks. Identical growth conditions were programmed into each chamber. The Table below reports plant biomass accumulation. The A184A null plants did not differ from A184A transgenic plants. However the B027A transgenic plants significantly outperformed the corresponding null plants. Mean biomass production was 28% higher in the transgenic plants. Photoassimilation and respiration data collected during the second week of the study illustrate the physiological basis for the difference in biomass. FIG. 1 shows the B027A transgenic plants have a higher daily photoassimilation rate and respire less at night. Both metrics indicate that transgenics are putting more carbon into biomass. The difference in respiration was not expected.

TABLE 6

| | | | Average | | | | | |
|---|---|---|---|---|---|---|---|---|
| | | | initial dry | | Final dry weight | | | |
| | | | weight | Plant | (grams) | | Plant | |
| Construct | event | genotype | (grams) | number | Ave | StDev | number | P(n) |
| 19862 | A184A | null | 0.051 | 10 | 18.40 | 3.13 | 40 | 0.4706 |
| | | transgenic | 0.048 | 10 | 18.89 | 2.81 | 40 | |
| | B027A | null | 0.052 | 10 | 10.58 | 2.78 | 40 | 0.0000 |
| | | transgenic | 0.047 | 10 | 14.76 | 3.65 | 40 | |

F1 hybrid seed were germinated and genotyped. Plants were organized into transgenic and null groups. Each group was cultivated in a large hypobaric chamber at the Controlled Environment Systems Research Facility at the University of Guelph. Shoots were harvested, dried and weighed. Initial biomass was determined for seedlings shortly after genotyping and represent shoot mass at the time beginning of the study. Data are the mean±standard deviation for each group.
Taken together the data illustrate that mathematical modeling is a useful tool for developing strategies to improve plant performance.

All references cited herein, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

1. An expression cassette comprising at least three polynucleotides selected from the group consisting of a polynucleotide encoding a phosphoenolpyruvate carboxylase, a polynucleotide encoding a fructose-1,6-bisphosphate phosphatase, a polynucleotide encoding a NADP-malate dehydrogenase, a polynucleotide encoding a phosphoribulokinase, and a polynucleotide encoding a pyruvate orthophosphate dikinase.

2. The expression cassette of claim 1 wherein the expression cassette comprises a polynucleotide encoding a fructose-1,6-bisphosphate phosphatase, a polynucleotide encoding a phosphoribulokinase and a polynucleotide encoding a phosphoenolpyruvate carboxylase.

3. The expression cassette of claim 1 wherein the expression cassette comprises a polynucleotide encoding a fructose-1,6-bisphosphate phosphatase, a polynucleotide encoding a phosphoribulokinase, a polynucleotide encoding a pyruvate orthophosphate dikinase and a polynucleotide encoding a NADP-malate dehydrogenase.

4. The expression cassette of claim 1 wherein the polynucleotides encode polypeptides having at least 70%, 80%, 90% or 95% identity to SEQ ID NO. 1; SEQ ID NO. 2; SEQ ID NO: 3; SEQ ID NO. 4 or SEQ ID NO: 5.

5. The expression cassette of claim 1 wherein the polynucleotide encodes a polypeptide comprising SEQ ID NO. 1, SEQ ID NO. 2, and SEQ ID NO. 3.

6. The expression cassette of claim 1, wherein the expression cassette comprises the polypeptide of SEQ ID NO. 2, SEQ ID NO. 3, SEQ ID NO. 4, and SEQ ID NO. 5.

7. The expression cassette of claim 1, wherein the polynucleotides are operably linked to one or more light inducible promoters.

8. The expression cassette of claim 1, wherein the polynucleotides comprise SEQ ID NO. 6; SEQ ID NO. 7 and SEQ ID NO. 8.

9. The expression cassette of claim 1, wherein the polynucleotides comprise SEQ ID NO. 9; SEQ ID NO. 10; SEQ ID NO. 11 and SEQ ID NO. 12

10. A method for increasing biomass comprising
a. introducing the expression cassette of claim 7 into a plant cell;
b. growing the plant cell into a plant; and
c. selecting a transgenic plant having increased biomass.

11. The method of claim 10, wherein the plant is a C4 plant.

12. The method of claim 11, wherein the plant is selected from the group consisting of sugarcane, maize and sorghum.

13. The method of claim 12, wherein the plant is maize.

14. A method of making a transgenic plant comprising:
a. introducing the expression cassette of claim 7 into a plant cell;
b. growing the plant cell into a plant; and
c. selecting a plant comprising the expression cassette.

15. The method of claim 14, wherein the plant is a C4 plant.

16. The method of claim 15, wherein the plant is selected from the group consisting of sugarcane, maize and sorghum.

17. The method of claim 16, wherein the plant is maize.

18. A plant or plant part comprising the expression cassette of claim 1.

19. The plant or plant part of claim 18, wherein the plant part is a plant cell.

20. The plant or plant part of claim 18, wherein the plant part is a seed.

21. A plant or plant part made by the method of claim 14.


SEQUENCE LISTING (12 sequences)

SEQ ID NO: 1 - protein, 970 aa, Zea mays
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIAHRRRNSKLKKGGFADEGSATTESDIEETLKRLVSEVGKSPEEVFEALKNQTVDLVFTAHPTQSARRSLLQKNARIRNCLTQLNAKDITDDDKQELDEALQREIQAAFRTDEIRRAQPTPQAEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWPEDKRQEWLLSELRGKRPLLPPDLPQTDEIADVIGAFHVLAELPPDSFGPYIISMATAPSDVLAVELLQRECGVRQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEARFVEYFRSATPETEYGRMNIGSRPAKRRPGGGITTLRAIPWIFSWTQTRFHLPVWLGVGAAFKFAIDKDVRNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 2 - protein, 415 aa, Spinacia oleracea
MASIGPATTTAVKLRSSIFNPQSSTLSPSQQCITFTKSLHSFPTATRHNVASGVRCMAAVGEAATETKARTRSKYEIETLTGWLLKQEMAGVIDAELTIVLSSISLACKQIASLVQRAGISNLTGIQGAVNIQGEDQKKLDVVSNEVFSSCLRSSGRTGIIASEEEDVPVAVEESYSGNYIVVFDPLDGSSNIDAAVSTGSIFGIYSPNDECIVDSDHDDESQLSAEEQRCVVNVCQPGDNLLAAGYCMYSSSVIFVLTIGKGVYAFTLDPMYGEFVLTSEKIQIPKAGKIYSFNEGNYKMWDDKLKKYMDDLKEPGESQKPYSSRYIGSLVGDFHRTLLYGGIYGYPRDAKSKNGKLRLLYECAPMSFIVEQAGGKGSDGHQRILDIQPTEIHQRVPLYIGSVEEVEKLEKYLA

SEQ ID NO: 3 - protein, 402 aa, Spinacia oleracea
MAVCTVYTIPTTTHLGSSFNQNNKQVFFNYKRSSSSNNTLFTTRPSYVITCSQQQTIVIGLAADSGCGKSTFMRRLTSVFGGAAEPPKGGNPDSNTLISDTTTVICLDDFHSLDRNGRKVEKVTALDPKANDFDLMYEQVKALKEGKAVDKPIYNHVSGLLDPPELIQPPKILVIEGLHPMYDARVRELLDFSIYLDISNEVKFAWKIQRDMKERGHSLESIKASIESRKPDFDAYIDPQKQHADVVIEVLPTELIPDDDEGKVLRVRMIQKEGVKFFNPVYLFDEGSTISWIPCGRKLTCSYPGIKFSYGPDTFYGNEVTVVEMDGMFDRLDELIYVESHLSNLSTKFYGEVTQQMLKHQNFPGSNNGTGFFQTIIGLKIRDLFEQLVASRSTATATAAKA

SEQ ID NO: 4 - protein, 429 aa, Spinacia oleracea
MGLSTAYSPVGSHLAPAPLGHRRSAQLHRPRRALLATVRCSVDAAKQVQDGVATAEAPATRKDCFGVFCTTYDLKAEDKTKSWKKLVNIAVSGAAGMISNHLLFKLASGEVFGQDQPIALKLLGSERSFQALEGVAMELEDSLYPLLREVSIGIDPYEVFEDVDWALLIGAKPRGPGMERAALLDINGQIFADQGKALNAVASKNVKVLVVGNPCNTNALICLKNAPDIPAKNFHALTRLDENRAKCQLALKAGVFYDKVSNVTIWGNHSTTQVPDFLNAKIDGRPVKEVIKDTKWLEEEFTITVQKRGGALIQKWGRSSAASTAVSIADAIKSLVTPTPEGDWFSTGVYTTGNPYGIAEDIVFSMPCRSKGDGDYELATDVSMDDFLWERIKKSEAELLAEKKCVAHLTGEGNAYCDVPEDTMLPGEV

SEQ ID NO: 5 - protein, 948 aa, Sorghum bicolor
MAASVSGATICLQKPGSKSRRARDATSSFARRSVAAPRSPHAAKASVIRSDAGAGRGQHCAPLRAVVDAAPIATKKRVFYFGKGKSEGDKSMKELLGGKGANLAEMSSIGLSVPPGFTVSTEACKQYQDAGCILPAGLWAEILDGLQFVEEYMGATLGDPQRPLLLSVRSGAAVSMPGMMDTVLNLGLNDEVAAGLAAKSGERFAYDSFRRFLDMFGNVVMDIPRSLFEEKLEHMKESKGVKNDTDLTAADLKELVGQYKEVYLTAKGEPFPSDPKKQLELAVRAVFNSWESPRAKKYRSINQITGLVGTAVNVQSMVFGNMGNTSGTGVLFTRNPNTGEKKLYGEFLINAQGEDVVAGIRTPEDLDAMKDVMPQAYEELVENCNILESHYKEMQDIEFTVQENRLWMLQCRTGKRTGAGAVKIAVDMVSEGLVERRQAIKMVEPGHLDQLLHPQFENPALYKDKVIATGLPASPGAAVGQIVFTAEDAEAWHAQGKAAILVRAETSPEDVGGMHAAAGILTERGGMTSHAAVVARGWGKCCVSGCSGIRVNDAEKLVTIGSHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLPYQRSDFEGIFRAMDGLPVTIRLLDPPLHEFLPEGNIEDIVSELCAETGANQEDALARIEKLSEVNPMLGFRGCRLGISYPELTEMQARAIFEAAIAMTNQGVQVFPEIMVPLVGTPQELGHQVNVIKQTAEKVFANAGKTIGYKIGTMIEIPRAALIADQIAKEAEFFSFGTNDLTQMTFGYSRDDVGKFLPIYLSQGILQHDPFEVLDQKGVGQLIKMATEKGRAANPNLKVGICGEHGGEPSSVAFFDGVGLDYVSCSPFRVPIARLAAAQVVV

SEQ ID NO: 6 - DNA, 4825 bp, Artificial Sequence
  SoFBP in expression cassette ZmPRK-1
gaaatgagttttttctaatttactcagaatatgattttggagtattacatcattacgttgtccctcaaagactaaaaaagggactaaatcggttttgtctgtagtccctcaaaggatgattgaaatggactaaacgattatctttacggttcctgcccctcattgtgctacccctccttgcgatgtccaaataccaaagagactaagatgcatttggttgtaacgatgggacatgacgaaatgtgatgattcttaaataaggttgtctggtttagggtcaggggttagaacaaagctgtcctagtgttattaccagttgtccatcaaaattaaagagacgagatgagacggcagaacgtctttgtcctgcctatccctaggtatcccacaaccaagcgcaccctaagagagagcggtggttggttgaagaacaatatagtctctttttaatattatttaatgacccgcgataacttttaatcctgaaaaccaaacgactagtcccgcgactaaagtttaacagagggttaatgcaattcgctcgatgcatatacgacacacatgctttgggttggcatattccaaagaagaaaaaagaaaaaggaaaaaaagaaaagggaaattctctcaaaggtctaggacatcaggtgatgtggacgctgccaaagtcctgggctcctggctgacgcggatgcttacctggcgcacgcctacagagcggatgctgctttaccaagaacgtgcgtagcgcagcatgttacttggcgttggcgatcatcagcaacatcctcccaggtctcgcccccagccacccgtcattccctcatctgaaaccagccatccatgcgccgccacgtggagaaaccatatctgaatccatgcgaccccaaccaagctttcccacgatcgtccgtgggccatcactagtcaggccaggccactcagacatcctcagctaatcagcaataccgacaagtacggagatctcaaatacgtagtgtacgtctgatttagcagctaccagacgagcagtaagcaaaatgttttctgcatataactcgccattaaaccttgccaaggcaggtgttagaagcatcatcaggaaaaatggtcatgaaaaatattatagccttttctcagcaaggaaattaaatttagtgtccagtccagtggaggaataccgacagaatacactcgctgcgagaaaaagaaagaaggggaagaactcaatactgacaaaatacactactcgctgcgaaagaatgaaaggaagaactcaatactgacaaaatacactactcgctgcgaatagcgagtgaatgaaaggaaagtgaatgaaaggaagaactcgaagctgacaaaatacacactctcgctgcgattgaatggaaggaaaacgaatgaagggaagaactcgaagctgacaaaatacactcgctgggattggtagaaaggaagaactcattttcagctcattattataagctgtcctcgctattacgagggggaaacaaaaacaaaacgaaaaatagggacacgccacatcatcgccatcctcatttcgtcctgttatctcgtagctccacagtccacacccaccatcccgttctccctctcttctcctctccaaggtccctgccacccacacaaggcttggactcttgggccggccgggggggaagaagacaagacaaacgcagccgccggcttgtaggcgatctgcagcgcgcacaccaccaccatctccctgcgctcccctagcacgacgaccgtctcgaacgcggcagctggcttggtgcagaagcaagtcatcttcttgaccagcatcaacaggaggagcggcagcagaaggcgtggaggaggggtgagcaggaccttactccaggtctcgtgctccgccgacggcaacaagccagtggtgatcggcctggcggcggactccgggtgcggcaagagcaccttcatccgccggctcaccagcgtcttcggcggcgccgcggagccgcccaggggcgggaacccggactccaacacgctcatcagcgacaccacgaccgtgattagcctcgacgactaccactccctggacaggaccggcaggaaggagaagggcgtcaccgcgctcgacccgagggccaacaacttcgacctcaagtaggagcaggtgaaggcgatcaagcaaggccaggcggtccagaagcccatctacaaccacgtcaccggcctcctcgacccgccggagcttatcacgccgcccaagatctttgtcatcgaaggtctgcacccaatgtaagctcaggttctatatatgtgcccgtgtgcatgcatgctccgacccacttctgctgctacatacatacatacacataccccggtgctcaattctatatatcagagtgttgtgtgtgctgtgctcaatggaagtaacaagaaggttgtcttacaagccatgacagctacttttgtttgcttaaaccacagcttcgacgagcgtgtttgtcgaacaacaacaaacaacaaacaacaaagtcgaacaacaacaaacaacaaacaacaaagtcgaccaaaaccatggcttctatcggcccagctaccaccaccgctgtgaagctgaggtccagcatcttcaacccgcagagcagcaccctgagcccatctcagcagtgcatcaccttcaccaagagcctgcacagcttcccaaccgctaccaggcataacgtggcctctggcgtgagatgcatggctgctgttggcgaggctgccactgagactaaggctaggaccaggtccaagtacgagatcgagactctgaccggctggctgctgaagcaagagatggctggtgtgatcgacgccgagctgactatcgtgctgagcagcatcagcctggcctgcaagcagatcgcttctctggttcagagggccggcatctctaacctgactggcattcagggcgccgtgaacattcagggcgaggaccaaaagaagctggacgtcgtcagcaacgaggtgttcagcagctgcctgaggtcatctggcaggaccggcatcattgctagcgaggaggaggacgtcccagttgctgttgaggagagctacagcggcaactacatcgtggtgttcgacccactggacggcagctctaacatcgacgctgctgtgagcaccggcagcatcttcggcatctacagcccaaacgacgagtgcatcgtggactctgaccacgacgacgagagccagctttctgctgaggagcagcgctgcgtggtgaacgtttgccagccaggcgataacctgctggctgctggctactgcatgtacagcagcagcgtgatcttcgtgctgaccatcggcaagggcgtgtacgctttcaccctggatccgatgtacggcgagttcgtgctcaccagcgagaagatccagatcccaaaggccggcaagatctacagcttcaacgagggcaactacaagatgtgggacgacaagctgaagaagtacatggacgacctgaaggagccgggcgagtctcagaagccatacagctctcgctacatcggcagcctggtgggcgatttccataggactctgctgtacggcggcatctacggctacccaagggacgctaagagcaagaacggcaagctgaggctgctgtacgagtgcgctccgatgagcttcatcgttgagcaagctggcggcaagggctcagatggccatcaaaggatcctggacatccagccaaccgagatccaccagagggtgccactgtacatcggctccgttgaggaggtcgagaagctcgagaagtacctggcctgagagctctggcccgcgtgcattcagatgtcctaaaacgggacaggcctcttcaaactcgacgcacgtctgttggggatatatgcatgggcagcatggcgaggaactaggagcctaggaggatgtggaagaaacgtcatttgcagtgctcaggaaaacgtgcagcacttgtttagatgtgtgccttcttccatgcttcattgcagaaagaaatcaagtgcctctactactatcaggtactcctattcaagtgtaggagacgaatccataccacttccattgttggttattgtttctctgacccggagccaagaacagtcaacaaggacccgaggttgaacatctctttttatggactactggagagtaacaacatgtccgtttggttttaattagtactggattggactgcttctacagtactttgtctttatggattatagctgtagtagtcggttttaattcgtactggattggactgcttccacagtattttatctttatgcattgtagctgcagtagtccgaacaactggttttaatccgaggagagcattaatgttcttgccatctagcaattgaaaaccatagcaggcaaacaaaaaaaatcaaaattactcgtcgtttcaatatcacaaacggaaactgtaaaagcaagcaacaatcaatacagcagctgaacacatatcactccgttgtggttctacattttcatacaagcatatactactactagtaccgttccggccatcaaaacaagagccgtgggtaaacccagacctgccactagtacaatttggctatatacaagcggtaggctttttacatcacatgcggttcggttagaaaaccgcctgtgatgtcccaggcggttcagtacgcctgtgatgtaatagtatcacaagcggtttttgtttaggaccgactgtggtgctctatccttttcacaaacggaccctaagaaaaaaccgcctgtgattgtaaaaatatgtaaatacaatttaaatatg

SEQ ID NO: 7 - DNA, 4293 bp, Artificial Sequence
  SoPRK in expression cassette ZmSBP
cccgtcagcagagtggatagggcacattaaatgctgaggcggcacatcgcctgccagtggagtggacagggctcatttaatgctgaggcggcacatcgcctgccagtggaatggacaggcgacgcgccttatccgcattaaatgcagaggccgcgcggcctagtggccttacgtttggctccgcccgctggcttacgtcacgcgcagtagaccatatggcaacatcgggtctccgcctgagcggggagcagaggcgtatgcggtattgttcggacacgtgtcggctccggacctccgtctggccttgattaaggtccgggtactctttgtccacgaacctcgcgaccctgttgtgagtggcccagaccctgcacaggagggtccgggacgcgtcccaggggtccgggcacgcctgtggaggttctggaccttacccggaggtccgctccgtacgcacaggggtctggtactttcccaagggggttcgaacccactgctgatgccttggagcatatcgtcttttctggccacgtggcgactccggagccatccgcgtggtcgggtcgggtgttgttcatcacgcaactagagatagccgcgtgggcaccgtatcttcatgctgtagtaaggggtacccctgtttcagagtaccgacatgaacgataggtggagatcgtgggtgcaatttatggtgtaaactattgtgggtgattcaccatcctagagtgatgaagaatcaacatgcagggagtgcttgatccttgcgctgatcaagaggagccacacccttgcgcggttgctccaaaaaagactagtggaaagcgtcgactttctgatacctcagaaaaacatcgtcgtgttcctaacacttcatttactttgaatatttactattgtataattaacttcttatatttagattactagaattgtcaagttagaataaggttagaacttaaggtgctaagcttatatgtgaatggtagaaaatattattgggcacaatgtggcaagtgagctatttgatagaatttaattattgcgaaaaagtttatcgtttaatttatatttttctcttgagtatcttgatcggccagaaacatagcattgtaaagtatatttgaagctctccaatatggttaaaattgaaaaaaaaaattgcacaactaggcgtatccagtgagaaaaggccttgccactctacgtatctgatgttgttaataatttcagaagtcgtcgtatataccaaggggtgtttaattgtcgtatatacgatgggatgcttaattgtcgtatatacgatggtatgatgaaacaactgacttaaacatcacactgaacaatttcagaaaacgatccatgccgtcgtatatatacgacaacaaaataccagaagcaaacctcccagacccaaggggaaataaacgggcctgcttctggtcgctagcttgggggcgctggagctgcagtgcgtaggcccgtccgatccgtggctcgtctcggcatggccacacaaaccacgaacggtcgtcgtgcaccgcagcgcggcccccccgttctatcttctccagctccaaatcgcgccatcgcggcggccgggttatcttgtccagacgtgcatcatatcctccgtgtgatccattcatccccgcgccgtgctagcttgctagttgcaagcaccagccgaccaccaaacggtagcgcacgcggacaatttaacagcatcaggtttaggccctgctgccgtcgtcgagcgcgcgggccaccgcacacctgaaagcaatcgagatcgtcgccacgcgctccccggcttgctgcgccgccgtgtccttctcccagtcgtacaggcccaaggtacgtacggcaccttcatatctcgtgactactgtacgtaagcggaaagtagcagcagctcgtcgcgcacacgtgcagaagccttaagtttgctgatgatgttgatgactggcgccacacgtgcggcaggcgtccaggccgccgtttgtcgaacaacaacaaacaacaaacaacaaagtcgaacaacaacaaacaacaaacaacaaagtcgaccaaaaccatggctgtgtgcaccgtgtacaccatcccaaccaccacccacctgggctctagcttcaaccagaacaacaagcaggttttcttcaactacaagaggtccagcagcagcaacaacaccctgttcaccaccaggccgagctacgtgatcacttgctctcagcagcagactatcgtgatcggcctggctgctgattctggctgcggcaagtctaccttcatgaggcgcctgacctctgttttcggcggtgctgctgagccaccaaagggcggcaacccagatagcaacaccctgatcagcgacaccaccaccgtgatctgcctggacgacttccacagcctggataggaacggccgcaaggttgagaaggtgaccgctctggacccgaaggctaacgacttcgacctgatgtacgagcaggtcaaggccctgaaggagggcaaggctgtcgacaagccgatctacaaccacgtgtcaggcctgcttgacccaccagagcttatccagccgccgaagatcctggtgatcgagggcctgcacccaatgtacgacgctagggtgagagagctgctggacttcagcatctacctggacatcagcaacgaggtgaagttcgcctggaagatccagagggacatgaaggagaggggccacagcctcgagagcatcaaggctagcatcgagagccgcaagccagacttcgacgcctacatcgacccgcaaaagcagcacgctgacgtggtgattgaggtgctgccaaccgagctgatcccagatgacgatgagggcaaggtgctgagggtgaggatgatccagaaggagggcgtcaagttcttcaacccggtgtacctgttcgacgagggcagcaccatcagctggattccatgcggccgcaagctgacctgctcttacccaggcatcaagttcagctacggcccggataccttctacggcaacgaggttaccgtggtcgagatggacggcatgttcgacaggctggacgagctgatctacgtcgagagccacctgagcaacctgtccaccaagttctacggcgaggtgacccagcagatgctgaagcaccagaacttcccgggcagcaacaacgggactggcttcttccagaccatcatcggcctgaagatcagggacctgttcgagcagctggtggcttctaggtctaccgctaccgccactgccgctaaggcttaagagctcattactagaatccgggctcgtagatgctggagtacacagtacagggaaattgcccacttttttcatcaacttaagtttttagattaaacttttttgaaacaatcagacaggagatctgtcttatatattgatgaggagaaagatgcccaaaggcaaaaaaaaaaaagtcgatacaataacaagtccatcagctgctagaacagcctcccaaccgcaaaccaaaaaacaacccacgactagcatcctatctaagttgaagccaaaaagtagtcaagtgcctcgcctggaccgccatccagactgtcgcctcacatttaatgaggttacaatctactggctactagaaaacatgcaatcaaaagtactcgtatttctttcctaatatattgtcccgttgacaaggatcagcaacattctaaagcctttttctattacagcccaacaacatagccaattctcccaccaatgcatcaacgtgggagataactcctagctggatggcttcataactccagggtacctacacatagagcacaagttagggtatggggccaatttctagaagctaaacggcccagtctaaatacaattttgaattgcttagctgaaaaacttgctctttggaacgcccaggtatgagtctgtgcaaatcgaggcgaaaaattacgccttatatgccgatttcactgtgtatcggtggtctgcaatgaatttctagctgaagatatctttggcctctggatctaaatggatcttttgaacttcgaaccaaaaaaattgaagaactcatgaaaacggtgagggtgtaattactttgaccaagcagagcgagatccacgatctagacattgtcttttaccgcctctaccaatgatttgctcttgtttcttgatatagtaaagagcctaagtgccacgtccttcagtctcagcccttctgccgaaactctgttccagaagagtactttagaaccatcagtacatcaccaatcctaatatccgtcg

SEQ ID NO: 8 - DNA, 6555 bp, Artificial Sequence
  ZmPepC in expression cassette ZmPGK
gtacatgactttcttttgatggatgtaatatttttcatattcttttgcatttcaactttattgtgattttctgttgcatcgcctctcagtataaaactgtcgaaatgtaatccttccaaaatcatactattacctaaaagctaaaaacgatatgtttgatccagcaatgttctgtctccatattccctgtcatggtgcacttattaaaaatgcagcccacttttactttttacatctggagaatatgactaagaatctggttttacttgattcttgacttgtagatacctttttcttcgtatgagaccccacaaactgcgtcaaccccgacccggccaccacgccgccataccctcacagtacttgcatttgtttcatagaaacaatctactgttcctcgcaagacagaagtttattttgtattgtaaggttaaccttcatttatttttttttcaaatggtgaaattctggaatcaatagtatgtgtttgtttgatttggagacatctggattatttttaggcgtattgtgtgtctggggtttgcgtttttttgtttagtaccatagatgtaattctgttatttggtgggtctcatcctccctttacaggaaggcttgtacttcagacattcttttctttcttataaatacaaagatttacgactattgcaagttagaggtaaaaatagtgtgtttgtgcaagctcaaatattttcttataatagtataacacacatttgtacataagttattgtggtattatatgtttacgttgcaacgcacgggcactcacctagtatatgaagaagaagagtaagatttctcgatgcaaatatgcaagatagaaagaactcgtggccaaggtccctgacggctgccgctttcacaatggtctgatctcggactctgccacagcagcggcttgaccagcactaagcagaatagaacccagcgctggcttgttcgttttgatcttgaattgggtgggattgaaaaaaacgacagccgcagcttcttcttccagtgcggctgcagccgaacacagatagacgacggcctgttctgttccggtagggaattcaccttaggcgagaacgcggccggctgcaaagcttggcgagtatggagtaaaacttattttttgagggctgccgcctttggacaaatccagtaaactcaccgagtttcggaaatgtgggactgagaagggacggcgatcccagatcacacagaggacaggggaaaacgaagccaccgagcccccacacgtcgccatccatcgccgtaatcgatcaccgccgtctcctcccccacacacccaccggaaccgtcgtcctgacctctcgccagcgataagcaaatctcctccccactttatcgtccacaaagccttcttcccgccctcccgaatcgctccctctctgtccctgcgctccagccgccgccgtcgcctccgccccccgaatcccataagcgtccgcggccgcccctccaacctccctctccctcgcggcccgcgcggccaccagggcagccgccgccgccgccgccccgctgcgcaggggaggcctcgccacggcgtgccagccggcacggtctctggctttcgcggcgggcgacgcgcggctcgcggtccacgtcgcgtcgcgtagccggcaggcgttctccgggcgtggcacgcgggccatagccaccatagcgaagaagagcgtaggggaactcacggaggccgacctccaggggaagcgcgtcttcgtgcgcgccgacctcaacgtgccgctcgacgagaaccagaacatcaccgacgacacccgcatccgggccgccatccccaccatctagtacatcctcagcaagggcgccaaggtcatcctctcaagccacttggtgagttcccggcgtccgaccttcccatatccacgctcttcacactatgtaggaattcagtactccttggattcaggtctttgtgataatctgatttgctcattttatttgtcgcccgctagttcatttttgaactaaaccgcgacaaataaagaagaacggagggagtacatacatatggaccctagctattagttgtgattttgcttcccatgctatatgattttagcttatcttcaacatagctaactatcagtatatcaattctattttcgtttttgggcacaaactggtaatttctgcaaaggtgaaagatacttattttaggaaaaaagaacttacataagtagggaaaaactgctcttttaattcagaatctgtttgtgactccaatttagaaaattggactctgtaactgttgctcttcgcatacactcacaagtcacaatgtagcagccaaggacctgcataggatattgtttatttaaagttctggttttgtatatacagattggctattagttgcagattttcttattgggttcaatgataattttatgaaagatttgctgaaccaatatatttatctcagattgctgcttaataatcttttcatccagtcatgattaatatcctcccttttgctctggatgtgcagggtcgccctaaggtatttagtcgaacacaattacgtcgaacaacaacaaacaacaaacaacaaagtcgaacacaattacgtcgaccaaaaccatggcctctactaaggctccaggcccaggcgagaagcaccactctatcgatgctcagctgaggcagctggtgccaggcaaggtgtcagaggacgataagctgatcgagtacgacgccctgctggtggatcgcttcctgaacatcctgcaggatctgcacggcccatctctgcgcgagttcgttcaagagtgctacgaggtgagcgccgactacgagggcaagggcgatacaactaagctgggcgagcttggcgctaagctgactggccttgctccagctgacgctatcctggtggctagcagcatcctgcacatgctgaacctggccaacctggctgaggaggtgcagattgctcacaggcgccgcaacagcaagcttaagaagggcggcttcgctgacgagggctctgctactaccgagtctgacatcgaggagactctgaagaggctggtgagcgaggtgggcaagtctccagaggaggtgttcgaggccctgaagaaccagaccgtggacctggtgttcaccgctcatccaactcagagcgctaggcgctctctgctgcagaagaacgctaggatccgcaactgcctgacccagctgaacgccaaggacatcaccgacgacgacaagcaagagctggacgaggctctgcagagagagatccaggctgctttcaggaccgacgagatcagaagggctcagccaactccacaggccgagatgaggtacggcatgagctacatccacgagactgtgtggaagggcgtgccaaagttcctgagaagggtggacaccgccctcaagaacatcggcatcaacgagaggctgccgtacaacgtgagcctgatcaggttcagcagctggatgggcggcgatagggatggcaacccaagggttaccccagaggtgaccagggatgtgtgcctgctggctaggatgatggccgccaacctgtacatcgaccagatcgaggagctgatgttcgagctgagcatgtggcgctgcaacgatgagctgagggttagggctgaggagctgcactctagcagcggctctaaggtgaccaagtactacatcgagttctggaagcagatcccgccgaacgagccgtacagggttatccttggccacgtgagggacaagctgtacaacaccagagagagggccaggcatctgctggcttcaggcgtgtcagagatcagcgctgagagcagcttcaccagcatcgaggagttcctcgagccactcgagctgtgctacaagtctctgtgcgactgcggcgacaaggctatcgctgatggctctctgctggatctgctgaggcaggttttcaccttcggcctgagcctggtgaagctggacatcaggcaagagagcgagaggcacaccgacgtgatcgatgctatcaccacccatctgggcatcggcagctacagagagtggccagaggacaagaggcaagagtggctgctgtctgagctgagaggcaagaggccactgctgccaccagatctgccacagaccgatgagatcgctgacgtgatcggcgctttccatgtgctggctgagctgcctccagactctttcggcccgtacatcatcagcatggccaccgctccaagcgacgttctggctgttgagcttcttcaacgcgagtgcggcgtgaggcagccacttccagtggttccactgttcgagaggctggctgacctgcaaagcgctccagcttctgtcgagaggctgttcagcgtggactggtacatggacaggatcaagggcaagcagcaggtcatggtgggctactctgactctggcaaggatgctggcaggctgtctgctgcttggcagctttacagggcccaagaggagatggcccaggttgccaagaggtacggcgtgaagctgactctgttccacggcagaggcggcactgttggcagaggtggtggcccaactcatctggctatccttagccagccgccggataccatcaacggctctatcagggtgaccgtgcagggcgaggtgatcgagttctgcttcggcgaggagcacctgtgcttccagactctgcagaggttcaccgctgctaccctcgagcatggcatgcatccaccagtgagcccaaagccagagtggcgcaagctgatggacgagatggctgtggtggccactgaggagtacagatccgtggtggtgaaggaggcccgcttcgtcgagtacttcaggtctgctaccccagagactgagtacggcaggatgaacatcggcagcaggccagctaagagaaggccaggcggtggcatcactactcttagggctatcccgtggatcttcagctggacccagaccaggttccaccttccagtgtggcttggcgttggcgccgctttcaagttcgccatcgacaaggacgtgaggaacttccaggtgctgaaggagatgtacaacgagtggccgttcttcagggtgaccctggatctgctcgagatggtgttcgctaagggcgaccctggcattgctggcctgtacgatgagctgctggtggctgaggagttgaagccattcggcaagcagctgagggacaagtacgtcgagactcaacagctgctgctgcagatcgctggccacaaggatatcctcgagggcgacccattcctgaagcagggcctggttctgaggaacccgtacatcaccaccctgaacgtgttccaggcctacaccctgaagaggatccgcgacccgaacttcaaggtgacaccacagccgccactgagcaaggagttcgcagacgagaacaagccagccggcctcgtgaagctgaacccagcttctgagtacccaccaggcctcgaggataccctgatcctgaccatgaagggcattgccgctggcatgcagaacactggctgagagctcagcatgctttcattttgtttcgtcttcgtcttcacgtgccgttgtatacttgctacattctcgcttgcacttgcacctcctcagccgctcgcccgaaatgtaagagaccaatgttttatagagctaatggaaatcgtttgaacaacgacgaccctaatagtatgtgatttaccgagtgatctttcctcggtaacgtaactagtgatataaaaaacattcaaaggcaatcttggctattcactttgtgcaccaggactagcttcgctgagcaaggtgtgaattttcttttgttcttttctttgccagagaagcaaactctagcgtgcgctgatgccccgtgggaagctagatgtcacgttacggaggtctgctaccgaaaatttctggaccttggcattgtaaaatttctctcttgtctcaggcactagctggaaaattttcgctttagttcctctatttgagctaatggaaatcgccgttgatgccctcttcgccgcccggacgagtggtcttcatcgtgcccacaatcgctgtctcgactccccccgatcgccatctaataagcaggacgctgtgctgagctgccggtctctgttgtcaagaacctgtaaccatttaattgcaagggaaaataacagaggatcaattccgatgctttgcagacctgttggctgttggtccaccctgtgttgcatatacaccaggccagggcgctcggaacatgggcaagtagtatcggctccactgacatattgcaactctgtggccactcatcagcaggcgattaaaagagacagcaaaccatgctggactacacattccgcagacatccaacacaattgagagctatacgacagacagcatagaaccgacatcctcatgttcatacacagaatgttatgtgtcacacaaaacactgtgacaaagaaagttcatacgcagggcagctctccagacacacgtggcagaaaacaaggttttctgaaggctggagctggg

SEQ ID NO: 9 - DNA, 4787 bp, Artificial Sequence
  SoFBP in expression cassette ZmPRK-2
cctggtctacacgactagaatttggatttagcatgctcaacctttgaaaatgttactctgctcatcccttttatagtgtaagggaggagagaggttacatcaaccttggattccaccagctaagacctaattgtctcattaaaatgtttgcatatatagaagcaatgagcatttgtgactaaatgcctcgattggggcacatggctagctcaacacagtcatagttagtcattgatagctcacgagagataggtatatatatatccttgcaagacacgtctaaatataaatctttatgatgaccctattttcactactactcgtgccacatgtcttgcatctaccaaaatagataattctaagagaacaagtctctttgccttatagaataaactaagcattttaaattaatgtgaacatataacctatattaacaaacaaactaaaactaaaactaaatctaaaactaaatattattactaacaaactaaaacctaaaaaccaaagatactaaccaagattaacctaaacataaatatgccaactttccaactaaataactaatctaaatatagagcacatatacaactacattcaaccaaagtttttatcgtgtttgacctacctaaaatccctaacatctctatcaaaatatattttccctttaaaccctagcaatcaatgacaggtcagtcgcaccatacggtatggtatagtatagcgcctggttagttttgaaaaataatttttaaataattagaaatgtttttttaaaaaaactcttttagaattggaaccggggccaagaacatacatatggtgcgcagcgcagcgttgcatgttacggccacgaaccacgatcatcaacaccatgctcccaaagacctaccaggtctcgcgcctccagcctacccatcattccctcatctgcagccagccatccttgcgacgccacgtggtgaaaccatatctatattcatgaaacctcaaccaagctttcccacgagcgtccttggccatcactagtcaggcatcagctaatcagcaatgggataaaaaaaagcacaagtgaggtccaggccaaaaaatacagacaagtacggaaatctcaaatacgtacttccactgtacgccgcatttaactcgctatatgaaacctcgccaaggcatgttagaagcatcaacaggaagaatggtcgtgaaaatcttaaagctttctcacaagaaaaatttagtgtccagaggaggattggaggaatactgacaaaatacgcttgctgcgtatgaatgaaaggaggaattcaatactgacaagatacaatctatatgcgaatgaatgaaaggaagaactcaatactgacaaaatacactcgctgctaatgaatgaaaggaagaactcaatactgacaaaatacattcgcggagttgcggtgaatgaatgaaaggagaaactcaatactgacaaaatacactcgctgcaaatgaatggagaactcattttcagctcactacaagctgcccttgatattatcagaagaaaaaaaagaatgtgaaaaatagggacaccacatcattgccatccgcatttcgtcctctgattcttgttatcttgtagctccacatccaccatcccactctccctattcttcttctcttcaagtgccactcccatccaccacaaggcttggcttggtgggaagaagacaaacgccggcacgcgcacgcagacacgaaggcgatctgcagcgcgcacactaccacctccctgcgctccccttgcacgaccgtctcgaacgcaggtctgaggcagaagcaagtcatcttcgtcaccagcaacaggaggagcggcggcggcaggaggcacggaggggcaaggagcttccaggtctcgtgctccgtcgacaagccggtggtgattggcctggcggcagactcagggtgcggcaagagcaccttctaacgccggctcaccagcgtcttcggtggcgccgcggagccgcccaagggcgggaacccggactccaacacgctcatcagtgacaccacgacagtgatttgcctcgacgactaccattccctggacaggaacggcaggaaggagaaaggtgtgaccgccctcgaccctagggccaacaactttgatctcaagtttgagcaggtgaaggcgatcaaggaaggccaggcagtcgagaagcccatctacaaccaagtcactggcctcctcgaccctccggagcttatcgcgccaccaaagattttcgtcattgaaggtctgcacccattgtaagctcacgctctgtgtgcccttgttccactcactacgctactgcatatataccccggtcaattcttccacacttggctctatttgattagttgtcaggtacatggcgacaataagctttcccggcataaactctaacaagtggaagtaacaagattttgttttcttacaccaggttcgtagagcgagttttacaacaattaccaacaacaacaaacaacaaacaacattacaattagtatttacataaaccaaaaccatggcttctatcggcccagctaccaccaccgctgtgaagctgaggtccagcatcttcaacccgcagagcagcaccctgagcccatctcagcagtgcatcaccttcaccaagagcctgcacagcttcccaaccgctaccaggcataacgtggcctctggcgtgagatgcatggctgctgttggcgaggctgccactgagactaaggctaggaccaggtccaagtacgagatcgagactctgaccggctggctgctgaagcaagagatggctggtgtgatcgacgccgagctgactatcgtgctgagcagcatcagcctggcctgcaagcagatcgcttctctggttcagagggccggcatctctaacctgactggcattcagggcgccgtgaacattcagggcgaggaccaaaagaagctggacgtcgtcagcaacgaggtgttcagcagctgcctgaggtcatctggcaggaccggcatcattgctagcgaggaggaggacgtcccagttgctgttgaggagagctacagcggcaactacatcgtggtgttcgacccactggacggcagctctaacatcgacgctgctgtgagcaccggcagcatcttcggcatctacagcccaaacgacgagtgcatcgtggactctgaccacgacgacgagagccagctttctgctgaggagcagcgctgcgtggtgaacgtttgccagccaggcgataacctgctggctgctggctactgcatgtacagcagcagcgtgatcttcgtgctgaccatcggcaagggcgtgtacgctttcaccctggatccgatgtacggcgagttcgtgctcaccagcgagaagatccagatcccaaaggccggcaagatctacagcttcaacgagggcaactacaagatgtgggacgacaagctgaagaagtacatggacgacctgaaggagccgggcgagtctcagaagccatacagctctcgctacatcggcagcctggtgggcgatttccataggactctgctgtacggcggcatctacggctacccaagggacgctaagagcaagaacggcaagctgaggctgctgtacgagtgcgctccgatgagcttcatcgttgagcaagctggcggcaagggctcagatggccatcaaaggatcctggacatccagccaaccgagatccaccagagggtgccactgtacatcggctccgttgaggaggtcgagaagctcgagaagtacctggcctgagagctccgatctatgcattcagatgtcctaaaactacagctctccgaactcaatgggagtaacaaccttcgcatctgttgggatatatggcgagctaggaggtatagaaatgtcattgcagaactcaggaaaacgtgcaatggaatttcttgaaatccctcttgaagagagtgagaactcgatagatcaagaatcaccacacgttgtattaatcgtatggtataatatttatacatgtacaggatgagctatgcatactggcgagcgttggcagtctgcggcgtagcgtgagcggagtgtgttagcccttttccaaacctctaaaattaatagttagtagctaaaattagctaaaaagtttaaaacggatcagctaatgaaccagtttattgttagctatacttctcatatagctattagttggtagttgtttcaacccagccaacaattttttagctctagaggtttaaaatagggccttaaacgggccgttgcggtgagtggctaagggggtgtttgtgtatttgtcaatttagagactacaataaaataaaatctagaaactaaaattagtctcaagaaaccaaattgttgtgcatgctaacaccccttagtctagacgactgagtagatgacaacgagactgttgtggaagttattgaataggatcattgatagtccttttatgaggatgcctgcaaaccatcgtgcactaggaatgtgcggcgcacgcatgtcctctagagcatctttatccctaacaaaatggatgtccaatacaatatgcttgccccctcggtgatgcacatggttttaggacaagtagatggaagaaacattatcacaatatgtgatggtcgccttagggacattgaagtgaagttcactaagaaaattgtgtagtcagacattggtgatcccacgatactcgaccttcgcactcgacctagagactgttggttgtcgcttcaataatggccggggcatgagagcatc

SEQ ID NO: 10 - DNA, 4350 bp, Artificial Sequence
  SoPRK in expression cassette ZmNADPME
atttgtcgggttcattattcgtctgattagttatctgcaccgtttcgtcctgagccaccacacacgttttgattttgtcagagtttatgttaaagacaccaaaaagcagaaaacattgcgtgccgatcaattggacgcaatggaaagaaaaaaaagactggtgaaaagattcaacttcgcgaagaattaaggcggcaagctcttgctttggcttatgtatgccatgctgccatgcacttcaaataaggctgtttatttataaagaggcagtggtggtacgatatgtttttttttggttgatttatacagtaagtacccaatgttttgaagtcattgcattgcattgggcgcccgatgttctagtgctttaacgaaagaaatgcaggcaagtttgcccacgcttcagtgccaccgcttccatccggcaacaggcaacaggcaacagccagtggggggtgttggtcgatctctgtggccgtccgctgatgctggtagttgactgcctccatccgcgtgacgacggataagatgacggtgcctaggcaagatagacgagctgaaacgctggcccaacccaaaatcgtatgggtagtatgctgcgtcttcttccagaggcggtagctagctagatatatgagcgagcacgccacggctgcgcggtacgtgttagcctctcttttgatcagtgatcggcaaccaaaggagcgggatgaggccgccccgcttttctatcggtgatcagtgatgagtagcaaaagaaacggggcggcgatcctttcaccaccgcctttgcgcgacttgattagtgggcaggaccacggcgtcaggctagctggtccgccaacgacagcgatttttagccaagctcatccagcggccctccctcctggttgaagaattgcgatgaaaaataggggcgtgctagtctcaacattacagcttcctttcacagccagaaaaaaaatcacaatgtccaaccaaaacatggagtcgtcacaaacttattccatatatatagctttccacgtacataggggcgtgttttggctaaggtgccacacgcggctgccgcatgaggccgaggcgcagtgttggaaattggcggcatcataaacgtgacgttgttttcaacgggaaattaacgtacgtagtggccccgtcacacgtgaaaagcccaaggaaaaacagcaactttcgtctgtgtcattcaaatatattttcctcgttgttttacatcatcaccagcaacatataaagataggaaatttgggtgtcctaattctcctaacgatggtatggaatggtaaaagctaaagcgtggtatggacgtatggtgtggtttagaacgaatggggggctagattaataacgcagcagtgcaccccactgactaaggatatgatcatcccgcccaacgaatgatcgatcatcccgtcggctacagcgggggaagcacgcagtcaatacccgtggtcggcagcccgcagcccgcagccagcagcccccgcagaccgcagaccgcgcagcagtacctccagccagccctccactccccgtccgtcccgacgtgcgcgtgcgccgcacacgcgcaagcgcaactgctcaaaaccgcaccgcgccgagccgcagccgccgaggcccctggctttccctttttatacccctcgccacccgcatccccctgctccatccccccctctccacactgccaactcgctccgaagagggaggaggacgacgccggtagccactgacactgccgcgccgcgccgctcccgtctcccctccctccgcggtaactagacgccaccaagctgtccacgcgcaccgccgccgtcgccgcctccgcgtcccccgcctccccggtacgttccggacggttccacgagcgcccggcccggcccaactaaccacctttcgacgccaccaccttccctccgctagcgactccctcccggtgcttctcccgcgcggtttgggcatcgcaggttgccaccgcctcatcgtttgggcttgtgtgtgtgtgtgtcgcagtggaagctgggaggacggatttaactcagtattcagaaacaacaaaagttcttctctacataaaattttcctattttagtgatcagtgaaggaaatcaagaaaaccatggctgtgtgcaccgtgtacaccatcccaaccaccacccacctgggctctagcttcaaccagaacaacaagcaggttttcttcaactacaagaggtccagcagcagcaacaacaccctgttcaccaccaggccgagctacgtgatcacttgctctcagcagcagactatcgtgatcggcctggctgctgattctggctgcggcaagtctaccttcatgaggcgcctgacctctgttttcggcggtgctgctgagccaccaaagggcggcaacccagatagcaacaccctgatcagcgacaccaccaccgtgatctgcctggacgacttccacagcctggataggaacggccgcaaggttgagaaggtgaccgctctggacccgaaggctaacgacttcgacctgatgtacgagcaggtcaaggccctgaaggagggcaaggctgtcgacaagccgatctacaaccacgtgtcaggcctgcttgacccaccagagcttatccagccgccgaagatcctggtgatcgagggcctgcacccaatgtacgacgctagggtgagagagctgctggacttcagcatctacctggacatcagcaacgaggtgaagttcgcctggaagatccagagggacatgaaggagaggggccacagcctcgagagcatcaaggctagcatcgagagccgcaagccagacttcgacgcctacatcgacccgcaaaagcagcacgctgacgtggtgattgaggtgctgccaaccgagctgatcccagatgacgatgagggcaaggtgctgagggtgaggatgatccagaaggagggcgtcaagttcttcaacccggtgtacctgttcgacgagggcagcaccatcagctggattccatgcggccgcaagctgacctgctcttacccaggcatcaagttcagctacggcccggataccttctacggcaacgaggttaccgtggtcgagatggacggcatgttcgacaggctggacgagctgatctacgtcgagagccacctgagcaacctgtccaccaagttctacggcgaggtgacccagcagatgctgaagcaccagaacttcccgggcagcaacaacgggactggcttcttccagaccatcatcggcctgaagatcagggacctgttcgagcagctggtggcttctaggtctaccgctaccgccactgccgctaaggcttaagagctctgctgcggggatcaattttgcagtaataaaaaatctatcaacgcggatggtactctgttgtttatagtccctgctgctaaccacccttgttgctggtgctgctggagaggcattgtacctgtccatgcatatatgatatatatatgttgtaacgttgtgaaagcaaacaatcttgggtaccaatgtttgttattctttcgctcgattatgatggtctgttatagtggctggacgagtcagatctccgtgatagggaatcaagatgaccaaatctaagccaaaccaaataactctgcaaaccatctagccttcagcacaaaccaagtgttgggggttggggtgggggggggggggagaagacacagagtttaacgtggaaaaacctcccccgatgtggagaagaaaaaaaaaccacggaaaaacagggtacaaaggagtctatttatataggcaaaggagataaagatagagtcaaatagtcttatccaacaaatctccccttgacgctaaatctataaaactgtttccccaaacaccactagtgcgctaagcttcacgaacacctatcaagtcaaggcaatgcttgaacttggtattagataatggctttgtaagcatgtctgcaggattttcatcagtatgtatcttgtccactttaggccttgttcggttattgatattccatgtggattgaagtgtattgggtgggattgggatggattttgacttgctatggatttaatccgactcaatcccacccaatccacatggattaacgcaaaaacgaacaagcccttaatcttgtcttcagcaacaatatcacaaatgaagtggtacttgatatcaatatgcttggtcttgaattggtacatgtcattcttggtcaaacatatagcactatgactatcacaataaaccttgatgacatcctgagaaactccaagttcagaaataagaccttgcatccaagtagcttctttaaccccttcagtagc

SEQ ID NO: 11 - DNA, 15959 bp, Artificial Sequence
  SbPPDK in expression cassette ZmPEPC
tagaggcaacccaagataggtgaaagataagcttcctttgtcacaattgaatattcgtgcaaggtggtccaactattattttgagatgtttattgagaccattgaggacctttgagtaattaactctcaacctagtagaaattcgttaccaactgggttgcataggatttcatgattaacagtgtgtttggtttagctgtgagttttctcctatgaaaagactgttgtgagaacaaaaagttgaaaatcgtttagttcaaactgttgtgagttatccactgtaaacaaattgtatattgtttatatacactatgtttaactatatctcttaatcaatatatacaattaaaaaactaaattcacatttgtgttcctaatattttttacaaataaatcattgttcgattccatttgtaatattttttattaaaattgtttttatttcatttattataaacacttaattgttttaatcctattttagtttcaatttattgtatctatttattaatataacgaacttcgataagaaacaaaagcaaggtcaaggtgttttttcagggctagtttgggagtccaaaaattggagggggttagaggggctaaaatctcattcttattcaaaattgaataaggaggggattttagcccctctaatcatcttcagttttgtggctcccaaactagccctcaaagtagatgtggaaaagttgaaccccttttattcagcttctagaagcaggtttgaaaaatagaaccaaacaaaccctaaaagtgtgtgaatttttaacaggtaatggcaggttaattattcacatctctttggtcatgtttaagaggctgaaaatagatcaattgcaagaacaaatagcagagtggataggggtggggaggggtcgtctccctatctgacctctctcctgcattggattgcctttctccgtactctatttaaaagtacaaatgaggtgccggattgatggagtgatatataagtttgatgtgtttttcacatacgtgacaagtattattgaaagagaacagttgcattgctactgtttggatatgggaaaactgagaattgtatcatgcgatggccgatcagttctttacttagctcgatgtaattaatgcacaatgttgatagtatgtcgaggatctagagatgtaatggtgttaggacacgtggttagctactaatataaatgtaaggtcaaaattcgatggtttattttctattttcaattacctagcattatctcatttctaattgtgtgataacaaatgcattagaccataattctgtaaatacgtacatttaagcacacagtctatattttaaaattcttctttttgtgtggatatcccaacccaaatccacctctctcctcaatccgtgtatcttcaccgctgccaagtgccaacaacacatcgcatcgtgcaaatctttgttggtttgtgcacggtcggcgccaatggaggagacacctgtacggtgcccttggtagaacaacatccttatccctatatgtatggtgcctttcgtagaatggcaccccttatccctacaatagccatgtatgcataccaagaattaaatatactttttcttgaaccacaataatttattatagcggcacttcttgttctggttgaacacttatttggaacaataaaatcccgagttcctaaccacaggttcactttttttccttatcctcctaggaaactaaattttaaattcataaatttaattgaaatgttaatgaaaacaaaaaaattatctacaaagacgactcttagccacagccgcctcactgcaccctcaaccacatcctgcaaacagacaccctcgccacatccctccagattcttccctccgatgcagcctacttgctaacagacgccctctccacatcctgcaaagcattcctccaaattcttgcgatcccccgaatccagcattaactgctaagggacgccctctccacatcctgctacccaattagccaacggaataacacaagaaggcaggtgagcagtgacaaagcacgtcaacagcaccgagccaagccaaaaaggagcaaggaggagcaagcccaagccgcagccgcagctctccaggtccccttgcgattgccgccagcagtagcagacacccctctccacatcccctccggccgctaacagcagcaagccaagccaaaaagaagcctcagccacagccggttccgttgcggttaccgccgatcacatgcccaaggccgcgcctttccaaacgccgagggccgcccgttcccgtgcacagccacacacacacccgcccgccaacgactccccatccctatttgaacccacccgcgcactgcattgatcaccaatcgcatcgcagcagcacgagcagcacgccgtgccgctccaaccgtctcgcttccctgcttagcttcccgccgcgccttggcgtcgaccaaggcacccggccccggcgagaagcaccactccatcgacgcgcagctccgtcagctggtcccaggcaaggtctccgaggacgacaagctcatcgagtacgaagcgctgctcgtcgaccgcttcctcaacatcctccaggacctccacgggcccagccttcgcgaatttgtaactaaccaccgccgcggcccatttcttcttcgaccggttgccgcctgcgcgcggcactggtcgtgtcgtgtgctcgctcgtctccctccggtgcttactactgtaatccttgcaggtccaggagtgctacgaggtaaaccatggcggcgtcggtttccggggccaccatctgccttcagaagcctggctccaaaagcaggagggccagggatgcgacctcctccttcgcgcgccgatcggtcgcggcgccgaggtccccgcacgccgccaaggcgagcgtcatccgctccgacgccggcgcgggacggggccagcattgcgcgccgctcagggccgtcgttgacgccgcgccgattgccacgaaaaaggtatataccttgcagctcttgtatcacaaactgatggaatttgcgaggcagccatgcttattggcccgagctagcattttattggccggatacatgttaattgccatgacgtgcatggccgcatgggtacgcgtatatatatatatatagggataaaattaaacgcacaggaacacaggtaaatatatacggacgaaaagtctgaaaattaaattaaaaccgcataatttaatattttcatgtatgcacgctaaagtcacaataatatacacatagaaaccggtctaatattcacttgcatgcatgccatgtgtgttaatatattaatatgcatatttggtggctaatatattaatattaacctaacataaggacatgtgattgttacgcatatgacacatagattgaaaacgggatagacacaagtccatcccgtatcaggatctcccaaagcaaaaacgaacagaaaaccagcctatcctaattatacacattcgaaaacagatttttgcaaatatagaaacgggacagaatttttgcgtcccattttcatccgtctaggtattccgtcccgttttcttacgtctaggtacgcatgcgcgcaccatcacacatccccggcatcgagcgcgagcacatgtcttcccaccaaggccaaggtgatgtcctcgtaagcatggaaatgaacaagtactgcttatttccgagcacactagcatattatggacaattccaacctggtgagcaagctggtctccaggactaacgctgcccaccaaggtttgatgtttccattttgttttgcttgggccggtttggggaccgttccgttgcgttacagcatctttagtccttatgagcacctttggttcaatttaaacacaattattagatggagcccggccaacttaacatagtaaggcccggtttggttcctagtagatgttagctatctaataattatctcttttagatccaaacatttatagatagtagactagctaactattagccaaacctttagataacaactatcttattagctagaccaaatcagataatagtagctaataggtggatcaacaacccaatcttataaattagctgagtatccaaacacttctcttagataataggtagctagctaggctagctaatattactagctatgtgctattaactaggacctaagatactctcctcaactggaaaaaaagggaggccagtgagggcctttgaacattgttcggttaatgtgaaacaatgttcacaactgatcctaacattgtccactatttagaactttttatgctagtagattgtaagaactcccaaacatattgttagattttttttgtccaaaaaacattcaatttttcatcataataagttcttcttttttactccaaacgtgggtctaactagatttgaggatattgggcttgggtcacaattggtctggcccaaaaagacccataaggtaggcctgttcaagttgttggaggtgtttgggttaggaaaacaggcatgagcccaaataaatagcatgagtgcacaattattttttatttctcgtagtgtaatgtaggccgatggcttgagcccaacccaaagcctggttcaaatagagggcccaatcatgtcaaatgcgaagtgaaatttctttctcaactcaagagcatctccaataattgtaaaaagtcattaataaactaatgagttttctaagttactaaaaaagttaaaaacatatatccctcctttgcaccacgagttctagactatttccaaataccacttttaaactatttttccttcctcttcaaaattctagaaaaaaaacatgtgacaacagggtttaaactctagtgtgtaacgtcccactagactatcctaccaccagaccagtggccctttcactttgaaacctttattacaacaagacaaactgccgcacgactatcaatatagagtgatgccgtctattttgtggcgatactaattacctcaggtaagattaatttaagaattagataaactgctgggcagtacgtttgcccctatactgcagagagagagagagagagagagagagtccatgcccaaggttttcgccaaaaccaggcgagcacaatgctatcatgctacaaccacggcaaagaatttttccaaggctcagttgtcagtacatccgcacatacatcaagaatgtgaacggaatcgagtatggaatccaccacggaatggatagtagacaggggcgccatcagatcagatgcaccttggcaacctagccatttgattatcacggtaggatcgctcggccatccggcaagtggcctcgctcgctctctttgtgatgacgcagagctaaaaaacaagaaccggaggtgtaccttttcttttgccctatctatgcggctaaatccaagaaatcacggggacttttgttggttcagcaaggttcgcttcacttggcacaatcaactggactagggacgtgttatacggcgcaattttctttgcccattcgtgccaatgagacaatggcatctcttcacttcccccacaaattctaccgacaataatcaggggcgaactctggcttcaaatagaagcagccatttaattactagcaacagtggtggcaggcagacatgctgatgagaggtagtactcctgcttgtggccattgtttgtcttgtctcagttttgtccagtgtttgtgtcccaggacttgcaagtttcaacttcactaatgtgtttgcgatgtgaggtcagatatggatcctaaggtcatgccctcataggacccatatatatggccataggagcaagatccaagagcagttgtatgactttatatccttcccaattcttttttttagagcacgccaatccttcccaattcttatgaatagggattttgattaacaaaaatcttcctatgcctttttagattttcaaatataaacatcctctattttggatttctcacttgccaaagatgaaaaaggagcggggatataactgtacgtgggatgtaatggcactgcctcggtgtggcaatgcaaataatccactaaccctaagacagcggataatgttttaaaatacatttttgtcaaaccgggaagctcactctaatttgagttgccccattttatttggttacaacatggaacacgttgtgcatataggtttttttttttggtccctctacgtaagattacctagctaaaaatctagtttttgaaaattttcaacggacgcactccgtttttccgttgtcatacgtagctagctagcggtccacctcattcactgatacgaagctcccaacggcgtactccttttgcccaactgaaacgacggcgtcatcagtcgtcacgtccactccaccatgtgttggccctccgtccctgtttggtgtttatacatacagtagaagaatttggttaaaaattgcaagtgacagcccaaaagtctatataaccattatttaccgtaccgtgcgacgcacacatggatggtatactgtagtagtttaccaaagccacgcagcagagagcggctcgcagcggcactcgattcgtgcgggcgcggggcgcgtgcaatgcaaattaaacgacggccatccgtgcgctctccgtctccttgtggcttttgtgcagtgcagtcgccccacatggacgcacggtggctctgcttctcgcccgaacgccgccgtgacgggaggcggagacagacgtacggacggccgcgcgccgcccgccggtgctgctctctctccccttgcccgccgggggcgccttcttcggtcgccctgagcgcgtagcgtgtcaccaacaaccaagcagttactatggactcacgcttccaaaagaaccgttttttttttctcatctactattgctgctgtccagctactcgtataactcaagtgacatcacagtagtcaagaaacgatcggattgcacgtaagctcctgatgcgagaagacgacaatttaaataaaaagggggaaatcaaatataatccttgccgagatcagggccgggtcgtgtagtgtacctgcgctgcgatcccatcatcgtctaacgcggacgcaacgacgagacccatcctgacacgaccaacaacgctatccgcttcgcttgctttgcgcacccatgcgtggccaaggcctgccggcgtgtgattgacagacagggtattttgttcgataaaaaagaataatatgcccgttcacaccttgagctagctacctgctggtggcaatttttcgtagcttggcttgcgaaaattccacatgttcatcccagcaatgcaaatgtctggccactagtccatctctggaacacacaatatacacaaaatgcgagtagcagagagagagagagagagagacctccgtccagtgtcgatcacaacaaattaaagctagtaaataaaagcctaacaacactgaagcaagcaagcaggcaaacgttcgtcagcgcgtcgtccttgcgaaacagaaagcgcgctagctagctgctgcaccgtacgtgtctaccgcgtcatgttgttgcattggtggcgcggtgcgtgcgtggatgcgtgttgacacgacagcgtgagtcacagaagcggcgccactggacgctagcagcattgatcaattcagttttcagttttttcttggctggacgatgcatcacgcacgcatggaacaagaaagggtgacacggccggcggtgccggtggtggttcttgcatgcattggactaaggctatgacgagcgcaggcgttgggtagtaggagtacaagtgtagttgggttggcatgccatttagttaccacttccaatttttccaagctttagttcatcgttctctcgtactccttacgtccttaagtaactttttttttgcttttacatcttatttgatcacttatcttattcaaaatttttatgtaaattataaaataaataaatcattattcaagtatctttaaaatataataagtcataacaagatagatagtatttatataaaagataaggcagacaatcaaacaagatatctaaaaaaaatacttattttagaatggagagagtacgaagcatcaagtacttagtactcctagtttggtgtgactgagggtcctgcggcaaattaaaatagcttcatggcattatatattatgacaaaatgcttcaaagacattttgttgtacaaaaagaagaatccgccacatcactagttttcttacactcagtttcactcagaaaaggttaattaaacagtgtgcgcagctaggggttattttggaaaacaaattaaatcaaaaccacctgcacgtacgtacgtacatacgagagcaagcagtgcacacatcaactagtttgtcctggatgtaacagaaaggggcgggccactgtaggtaagcaaaggcagtagtggctatggtgatgtggccgcgggcgtccggatatgttagctgggaaggggcaagcgtgtgttcacttgcttgacaccgtttctaactttgccaacaacaactactactatagtatacgtgtaaagctcatccagccatctgaacatgttgataaagaaaaaaagtcatcctaacacgatggatttttgctcaaccgattttgtgccaaaatgactcgtcatttattgtttacaaggggcaccccctgggtttgtgaaaaaaaagtgttacgtgcttgcaagttttgtgctgctgctgcgcacgctcgccctgtcacgtcatcactcgcagccaaggctcgggtgccgccgccgctgctataaatagagccgcgggggaggccctgcttcattcatcagtcacacacagcggctgtgttgtgtattttgtcactgatcagtgagtgatcagctgcctcgtgtttgtttcgtgtgtgtgctaatggcgcccgctcaatgtgaccgttcgcagagggtgttctacttcggcaagggcaagagcgagggcgacaagagcatgaaggaactggtgagtgagaagctgttttcttttttttttatgattaaattatgtgctgcatgctgttatgttacatacatacatacatacatatactgatggacggtggatcatcaatcagctgggtggcaagggcgcgaacctggcggagatgtcgagcatcgggctgtcggtgccgccggggttcacggtgtcgacggaggcgtgcaagcagtaccaggacgccgggtgcatcctcccggcggggctgtgggccgagatcctggacggcctgcagttcgtggaggagtacatgggcgccaccctcggcgacccgcagcggccgctcctgctctccgtccgctccggcgccgccgtgtccatgccaggcatgatggacaccgtgctcaacctcgggctcaacgacgaggtcgccgccggcctcgccgccaagagcggcgagcgcttcgcctacgactccttccgccgcttcctcgacatgttcggcaacgtcgtgagtatttccttccttcgaccagcacgtcgatcgtcggttccattttccgtccgtccggcttgtggtcaccgctactgcttgtcccactagcgatggatgcctagttttgcgcgcaatctcatcgacgacccatatcccatcgtccatcctccaaggctgccgtgtgccgtggcctggctgccctggcctggtgcttgctgccgccggacggatgggtccaccaaggctggagtttttgtctgtttgccaggcgaggtagggccagccgtcgtagggcgtgtgccgtttccttgggttaaacgaacgtggttggggccttgggccttgggggttgttggattattcggcccgtcaggccagtcatcatcgtgcctactacgatgtgtatcaaattcattcacgctcacgcgttggagacagcgattggactaagtgctcctcttgttttattaccaccaatactattatactaggaggagtattttcccagttgcaaacttgagctttggtctaaataaaattgctttaattttaatcaatttttttagaaaagtatactaacacacagattttaagaagattttttttttaaaaaaaaagataatttaatttaatgttgtggatgcaggtctatttttttgatgaacttcataaaaaaaactactttaacagttccatgacctgaggaagatgttttttgtcacacaaatgcaagttttgatgatgtaaaaaaaaagaagcgactttttgaggaaaaataaaaggtgaacatagtttcgtcagataataacaagaatcttgtaggccaatgcgcacaaatgtatgtatattccgcgcagaattaacctagaggtcgttgtcagtgttgaagctcacgctaccaactaactagattcatatacggaatgtaaacttggtttgtcgcttgtcggactcgaggaaagaacgatgatgactcaaattgctctcatcagattttgttttttccaaatgtaggaactgctgcttaattaatctacggatcctttatatttattgtttatttcctggccaggtcatggacattccccgctcactgttcgaagagaagcttgagcacatgaaggaatccaagggggtgaagaatgacactgacctcactgccgctgacctcaaggagcttgtgggtcagtacaaggaagtctaccttacagctaagggagagccattcccctcaggtaccatcctcagtcactcaacagtgtctgtatgaaacaaatctcctgatactactggagctgttttcctaattgtgcaccaaaatcatgtgctacaacacaaccttaataaattactgtgcttgccttgcttgcagaccccaagaagcagcttgagttggcagtgcgggctgtgttcaactcgtgggagagccccagggcaaagaagtacaggagcatcaaccagatcaccggcctggtcggcactgccgtgaacgtgcagtcgatggtgtttggcaacatgggcaacacttctggtactggcgtgctcttcactaggaaccctaacactggagagaagaagctgtatggcgagttcctgatcaatgctcaggtatacttatggtgacctcagtcaggcttccatccattgctagctcctgtttgatcctgaaccttaattagcttctgtgttctgttcatacatgactacttgacacatgtcctggttggtaaacgaaacatgctgtggaccggagtcaaataatgaatttgccatcatacaattttgtttcctatatattcagggtgaggatgtggttgctggaattagaaccccagaggatcttgatgccatgaaggacgtcatgccacaggcttatgaagagctagttgagaactgcaacatactggagagccactacaaagagatgcaggtacgtacattagcttttctgccttgagattctgcgagacaatgtagtactacttcctttgctatgaatgaactcaggctgacttggtttttgatatgtgtgtgatgcaggatatcgaattcactgttcaggagaacaggctgtggatgttgcagtgcagaacaggaaaacgtacaggcgcaggtgccgtaaagattgctgtggacatggttagcgagggccttgttgagcgccgtcaagcgattaagatggtagaaccaggccacctggaccagcttcttcatcctcaggtaatcaatcgtactaaccatgaacggcttatcaaatcaacgtgtcctagatgtttgtatattaattaagtagttgatatgcatgcattgatacctttttcctcttgtcttatggaaaaccagtttgagaacccagcgttatacaaggataaagttattgccacgggactgccagcctcacctggggctgctgtgggccagattgtgtttactgctgaggatgctgaagcatggcatgcccaggggaaagctgctattttggtaagtaatatccttttcatcctctgtaaaaaatagctcttctgtatttattcaggataatttttttcctttggaaatactcctatgtaggtgagggcggagaccagccctgaggatgttggtggcatgcacgcagctgctgggattcttacagaaaggggtggcatgacttcccatgctgctgtggtcgcccgtgggtgggggaaatgctgcgtctcgggatgctcaggcattcgcgtaaacgatgcggagaaggtgagctgagttcttgtttgcagaagccaaaacatgctgagaagtaaaagcttgtaatgagattgtgatatggatgcttactttgctatgtttatatttatagactcgtgacgatcggatcccatgtgctgcgcgaaggtgagtggctgtcgctgaatgggtcgactggtgaggtgatccttgggaagcagccgctttccccaccagcccttagtggtgatctgggaactttcatggcctgggtggatgatgttagaaagctcaaggtataatctcagaaatactaaccaatatgtactactccattagtcaaaacacagacataattttctttcaagttcagaccatgtactataatcattgtctatttagagatcagaaatgattgtttgtgcatatgttgtaggtcctggctaacgccgatacccctgatgatgcattgactgcgcgaaacaatggggcacaaggaattggattatgccggacagagcacatggtatctatttagtacttggttatagttacacccaacatattatggctaggatatatacttggacattttacactttctttatttaacttctttgttatagacaaggaaataaatagtttcatgttttttctcctgtactttggcagttctttgcttcagacgagaggattaaggctgtcaggcagatgattatggctcccacgcttgagctgaggcagcaggcgctcgaccgtctcttgccgtatcagaggtctgacttcgaaggcattttccgtgctatggatggtaagtgaaaatcacagtgcattcatttacagatttcgtattgaactggatgcactagttttactgaacaaaacaggagtaagcaaccttctctcaattaagcaaacattgactatgtattttcagaaaataaataactaaattaggcttgaacataagtgatagctactccagagtccagactgtatttttgaagtgtgcaggactggtttgaacttttttttttggtttgtgtttcaggactcccggtgaccatccgactcctggaccctcccctccacgagttccttccagaagggaacatcgaggacattgtaagtgaattatgtgctgagacgggagccaaccaggaggatgccctcgcgcgaattgaaaagctttcagaagtaaacccgatgcttggcttccgtgggtgcaggttggttttctgctattctatttttcacagaaaaatccgtttccacccgtgcctgatccatttggttgtatgctctctctgttcttttatagctgcatttttatggagtatttagcaggttttcttgtgttagtgaaatattgagaaagaacaaactcactgtacatttatgtataccttgactaatgttggaactgccaaaattttcaggcttggtatatcgtaccctgaattgacagagatgcaagcccgagccatctttgaagctgctatagcaatgtccaaccagggtgttcaagttttcccagagatcatggttcctcttgttggaacaccacaggcatgcatcttctttattttcgtattaatgtatatagtatctctgcagttcaaaatgacaaaatccatttgatgccaaaattgcataaacaactaatttctgtacacatttaagtttcgcttgtctggtcacttacacccagtttgtcttccaccaaattcattttcttgaaatactttttcgatattttaagtttgttacagtgacctgagtttcctttagacaactgacatttgatatttccaggaattgggacatcaagtgaatgttatcaaacaaactgctgagaaagttttcgccaatgcgggtaaaactattggctacaaaattggaactatgattgaaattcccagggcagctctaatcgctgatcaggtaggaaacaactaactcccttatttcagaaaatttaaaggatgactatttagattggctttgtagattatattttattcctatgctaatttgacatctttcattgttgttttggtttcacaacctggcagatagcaaaggaggctgagttcttctcttttggaacgaacgacctcacacagatgacttttggctacagcagggatgatgtgggaaagtttcttcccatttacctgtctcagggtatcctccaacatgacccctttgaggtaactgttgcaactctgtcaccctctcatctgaggtcatacttgtatttttctatcatttgcagatgtgtatctcctgtcgtcttgccattatgcatatcccccctgactttcgaatgtccataaacttatcaggttctcgaccagaagggagtgggccaactgattaagatggctacagagaagggccgcgcagctaaccctaacttgaaggttagtttcgggatctgtggacattgtttcgtttccttagaaaccaaggtttgattgtttggtgttgtatgtaaacaggtgggcatttgtggagaacatggtggagagccttcgtcagttgctttcttcgacggggttgggctggattacgtttcttgctcccctttcaggttggtcaagtgataaactcatgatccaatccaacaagtatatctctttacatcccggttatgttaacggcagcaaaatcttaactggtttttatatgaaataccttctgcagggttcccattgctaggctagctgcagctcaggtggttgtctgagagctcgcggcttctcttcactcacctgcagagtgcaccgcaataatcagcttccggatggtggcgttttgtcagttttggatggaaatgccgaactggcagcgtctgttttccctatgcatatgtaatttcctgcctctttatattcactcttgttgtcaagtccaagtggaaaatcttggcatattatacatattgtaataataaacatcgtacaatctgcatgctgttttgtaataattaattaatatcccagcccattggatggacttgtttaccaaggtgttacttcagtcaccctcttttagttgtgctaaacagtttctgattgatatttttttattagagtaacctagtgcatttacttaagagaaatgatatctagtggcactagtgattagtttgcaaggttgagaacttgttactcgctcctagaggttaacactagcaagtgattggagcttagggtttttcttgaatttcactagaaaaaatataaactagtatatcatgatatgcacttaagtctttttagtgttatctaccgacactcaaaaaggctttcttgctactcatttctcttactcctaaagcaaaaaaaaaatagccaaatgaccctccctctaacaataatcataatgaaatctcacctctcttttaggtgcaatatttttgtgggagtgggtctttttgggtgactgaggggctctaggaaggggatcagtagagatatctagcaaggtgtcaagtgtattcctgagatggttaggttttgaacaccacacatgtttctgaggaggggctctcataagctccttaggcactccatctctcacaataggggtggcagatttgggaggagtgagcttgacatgtttggggtggatgaaggtttctctgaaggttttaggccactacactcaccaaccttaccaacacaagtgacactcccatccttagcagcaaagcctaaccccgttcccccagttcccctcttgaactaactga

SEQ ID NO: 12 - DNA, 4932 bp, Artificial Sequence
  SbNADP-MD in expression cassette ZmPGK
gtacatgactttcttttgatggatgtaatatttttcatattcttttgcatttcaactttattgtgattttctgttgcatcgcctctcagtataaaactgtcgaaatgtaatccttccaaaatcatactattacctaaaagctaaaaacgatatgtttgatccagcaatgttctgtctccatattccctgtcatggtgcacttattaaaaatgcagcccacttttactttttacatctggagaatatgactaagaatctggttttacttgattcttgacttgtagatacctttttcttcgtatgagaccccacaaactgcgtcaaccccgacccggccaccacgccgccataccctcacagtacttgcatttgtttcatagaaacaatctactgttcctcgcaagacagaagtttattttgtattgtaaggttaaccttcatttatttttttttcaaatggtgaaattctggaatcaatagtatgtgtttgtttgatttggagacatctggattatttttaggcgtattgtgtgtctggggtttgcgtttttttgtttagtaccatagatgtaattctgttatttggtgggtctcatcctccctttacaggaaggcttgtacttcagacattcttttctttcttataaatacaaagatttacgactattgcaagttagaggtaaaaatagtgtgtttgtgcaagctcaaatattttcttataatagtataacacacatttgtacataagttattgtggtattatatgtttacgttgcaacgcacgggcactcacctagtatatgaagaagaagagtaagatttctcgatgcaaatatgcaagatagaaagaactcgtggccaaggtccctgacggctgccgctttcacaatggtctgatctcggactctgccacagcagcggcttgaccagcactaagcagaatagaacccagcgctggcttgttcgttttgatcttgaattgggtgggattgaaaaaaacgacagccgcagcttcttcttccagtgcggctgcagccgaacacagatagacgacggcctgttctgttccggtagggaattcaccttaggcgagaacgcggccggctgcaaagcttggcgagtatggagtaaaacttattttttgagggctgccgcctttggacaaatccagtaaactcaccgagtttcggaaatgtgggactgagaagggacggcgatcccagatcacacagaggacaggggaaaacgaagccaccgagcccccacacgtcgccatccatcgccgtaatcgatcaccgccgtctcctcccccacacacccaccggaaccgtcgtcctgacctctcgccagcgataagcaaatctcctccccactttatcgtccacaaagccttcttcccgccctcccgaatcgctccctctctgtccctgcgctccagccgccgccgtcgcctccgccccccgaatcccataagcgtccgcggccgcccctccaacctccctctccctcgcggcccgcgcggccaccagggcagccgccgccgccgccgccccgctgcgcaggggaggcctcgccacggcgtgccagccggcacggtctctggctttcgcggcgggcgacgcgcggctcgcggtccacgtcgcgtcgcgtagccggcaggcgttctccgggcgtggcacgcgggccatagccaccatagcgaagaagagcgtaggggaactcacggaggccgacctccaggggaagcgcgtcttcgtgcgcgccgacctcaacgtgccgctcgacgagaaccagaacatcaccgacgacacccgcatccgggccgccatccccaccatctagtacatcctcagcaagggcgccaaggtcatcctctcaagccacttggtgagttcccggcgtccgaccttcccatatccacgctcttcacactatgtaggaattcagtactccttggattcaggtctttgtgataatctgatttgctcattttatttgtcgcccgctagttcatttttgaactaaaccgcgacaaataaagaagaacggagggagtacatacatatggaccctagctattagttgtgattttgcttcccatgctatatgattttagcttatcttcaacatagctaactatcagtatatcaattctattttcgtttttgggcacaaactggtaatttctgcaaaggtgaaagatacttattttaggaaaaaagaacttacataagtagggaaaaactgctcttttaattcagaatctgtttgtgactccaatttagaaaattggactctgtaactgttgctcttcgcatacactcacaagtcacaatgtagcagccaaggacctgcataggatattgtttatttaaagttctggttttgtatatacagattggctattagttgcagattttcttattgggttcaatgataattttatgaaagatttgctgaaccaatatatttatctcagattgctgcttaataatcttttcatccagtcatgattaatatcctcccttttgctctggatgtgcagggtcgccctaaggtatttagtcgaacacaattacgtcgaacaacaacaaacaacaaacaacaaagtcgaacacaattacgtcgaccaaaaccatgggcctgagcactgcttactctccagtgggctctcacctggctccagctccacttggccacagaaggtctgctcagctgcacagaccaagaagggctctgctggctaccgtgaggtgctctgtggacgctgctaagcaggttcaggatggcgttgccactgctgaggctccagctacccgcaaggattgcttcggcgtgttctgcaccacctacgacctgaaggccgaggacaagaccaagagctggaagaagctggtcaacattgccgtgtctggcgctgctggcatgatctctaaccatctgctgttcaagctggccagcggcgaggttttcggccaggatcagccaatcgctctgaagcttctgggcagcgagagatctttccaggctcttgagggcgtggcaatggagcttgaggactctctgtacccactgctgcgcgaggtgagcatcggcattgatccgtacgaggtgttcgaggacgtggactgggctctgcttatcggcgctaagccaagaggcccaggcatggagagagctgctctgcttgacatcaacggccagatcttcgccgaccagggcaaggctctgaacgctgtggctagcaagaacgtgaaggtgctggtggtgggcaacccgtgcaacactaacgctctgatctgcctgaagaacgccccagacatcccggccaagaacttccatgctctgaccaggctggacgagaacagggctaagtgccagctggctctgaaggctggcgtgttctacgacaaggtgagcaacgtgaccatctggggcaaccactctactacccaggtgccggacttcctgaacgctaagatcgatggcaggccggtgaaggaggtgatcaaggataccaagtggctcgaggaggagttcaccatcaccgtgcaaaagagaggcggcgctctgattcagaagtggggcagaagctctgctgcttctaccgctgtgtctatcgccgacgccatcaagtctctggtgaccccaactccagagggcgactggttctctaccggcgtttacaccaccggcaacccatacggcattgccgaggacatcgtgttcagcatgccgtgcaggtctaagggcgacggcgattacgagctggctaccgacgtgtcaatggacgacttcctgtgggagaggatcaagaagtccgaggctgagctgctggccgagaagaagtgcgttgcccatcttactggcgagggcaacgcttactgcgacgttccagaggacaccatgctgccaggcgaggtttgagagctcagcatgctttcattttgtttcgtcttcgtcttcacgtgccgttgtatacttgctacattctcgcttgcacttgcacctcctcagccgctcgcccgaaatgtaagagaccaatgttttatagagctaatggaaatcgtttgaacaacgacgaccctaatagtatgtgatttaccgagtgatctttcctcggtaacgtaactagtgatataaaaaacattcaaaggcaatcttggctattcactttgtgcaccaggactagcttcgctgagcaaggtgtgaattttcttttgttcttttctttgccagagaagcaaactctagcgtgcgctgatgccccgtgggaagctagatgtcacgttacggaggtctgctaccgaaaatttctggaccttggcattgtaaaatttctctcttgtctcaggcactagctggaaaattttcgctttagttcctctatttgagctaatggaaatcgccgttgatgccctcttcgccgcccggacgagtggtcttcatcgtgcccacaatcgctgtctcgactccccccgatcgccatctaataagcaggacgctgtgctgagctgccggtctctgttgtcaagaacctgtaaccatttaattgcaagggaaaataacagaggatcaattccgatgctttgcagacctgttggctgttggtccaccctgtgttgcatatacaccaggccagggcgctcggaacatgggcaagtagtatcggctccactgacatattgcaactctgtggccactcatcagcaggcgattaaaagagacagcaaaccatgctggactacacattccgcagacatccaacacaattgagagctatacgacagacagcatagaaccgacatcctcatgttcatacacagaatgttatgtgtcacacaaaacactgtgacaaagaaagttcatacgcagggcagctctccagacacacgtggcagaaaacaaggttttctgaaggctggagctggg